(19) United States
(12) Patent Application Publication
(10) Pub. No.: US 2010/0034749 A1
(43) Pub. Date: Feb. 11, 2010

(54) Use of a Cationic Collodal Preparation for the Diagnosis and Treatment of Ocular Diseases

(75) Inventors: Brita Schulze, Walchensee (DE); Uwe Michaella, Weilheim (DE); Hansjürgen Agostini, Vorstetten (DE); Jing Hua, Freiburg (DE); Eric Guenzi, Dachau (DE); Gottfried Martin, Freiburg (DE); Lutz Hansen, March (DE)

Correspondence Address: MORGAN LEWIS & BOCKIUS LLP, 1111 PENNSYLVANIA AVENUE NW, WASHINGTON, DC, 20004, US

(73) Assignees: MediGene AG, Planegg (DE); Universitatsklinikum Freiburg, Freiburg (DE)

(21) Appl. No.: 12/308,748

(22) PCT Filed: Jul. 9, 2007
(86) PCT No.: PCT/EP2007/006071

§ 371 Date: Oct. 13, 2009

Related U.S. Application Data
(60) Provisional application No. 60/819,374, filed on Jul. 10, 2006.
(60) Provisional application No. 60/899,003, filed on Feb. 2, 2007.

(51) Int. Cl. A61K 49/00; A61K 9/127; A61K 31/573; A61K 31/337; A61K 31/60; A61K 38/20; A61P 27/02

(52) U.S. Cl. 424/9.6; 424/450; 514/179; 514/449; 514/177; 514/165; 424/85.2

(57) ABSTRACT

The present invention relates to cationic colloidal preparations and their use for the diagnosis and/or treatment of ocular diseases.

The present invention relates to cationic colloidal preparations and their use for the diagnosis and/or treatment of ocular diseases.

### INTRODUCTION

Ocular neovascularization in the form of retinal neovascularization (RNV) and choroidal neovascularization (CNV) are the most common causes of severe visual loss in the developed countries (Campochiaro, 2000).

The retina is supplied by two vascular beds. The inner retina is supplied by the retinal vessels and the completely avascular outer retina is supplied by the choroidal circulation. In adults, there is usually very few turnover of blood vessels, and angiogenesis, the growth of new blood vessels can lead to destructive neovascularization.

Age-related macular degeneration (AMD) is the major disease involving choroidal neovascularization and the most widespread “back of the eye” (BOE) disease and the major cause of vision loss in people over the age of 55. 14-24% of the population aged 65-74 years and 35% over 75 years in the U.S. are affected by AMD. The early stage of AMD is characterized by fatty deposits on the back of the retina, detectable as yellowish spots called drusen. This leads to the atrophy of retinal pigment epithelial (RPE) and retinal cells caused by toxic lipofuscin components. This form, known as atrophic or dry AMD, is the most prevalent form of late AMD (about 90% of AMD patients). Although it is responsible for only 10% of vision loss associated with the condition, it does predispose a person to developing the more severe wet form of late AMD.

Wet AMD is mainly characterized by CNV. The growing choroidal blood vessels break through the Bruch membrane under the retinal pigment epithelium (RPE) or into the subretinal space. These weak and underdeveloped vessels leak blood and fluid into the subretinal area causing damage to the macula. As a result of this process, patients can develop a detachment of the RPE and the neurosensory retina, a formation of a fibrovascular scar, and/or a vitreous hemorrhage/edema. The visual prognosis for most patients with wet AMD is poor, the disease is progressing rapidly.

Retinal neovascularization (RNV) is the one of the main pathologic effects in diabetic retinopathy (DR) and related diseases, a common cause of blindness in younger people. The prolonged periods of elevated glucose levels cause the deposition of modified fat and protein molecules within the capillaries leading to ischemic and hypoxic conditions. The resulting hypoxia leads to an upregulation of VEGF that promotes retinal neovascularization. In proliferative DR new blood vessels are growing into the retinal environment and, similar to wet AMD, are leaking blood and fluid into the retina and the vitreous. If the pathologic process of preproliferative leakage occurs predominantly within the macula area, this type is called diabetic maculopathy (DM), which often leads to the formation of a diabetic macula edema (DME).

Only recently it has been proposed, that inflammatory processes like complex deposition, complement activation and extravasation of neutrophils and macrophages are important mediators of the pathogenesis of ocular neovascularization diseases. Especially neutrophils may directly act as potent promoters of the angiogenic process by releasing pro-angiogenic factors like VEGF. Infiltration of neutrophils is usually triggered by the increased expression of chemotactic chemokines like IL-8 (or the murine counterpart KC), the expression of which has been found to be increased in a mouse model for CNV or cell cultures of UV irradiated RPE as an early model of AMD (Zhou 2006, Higgins 2003). It has also been suggested that IL-8 might also act as a direct promoter of angiogenesis. An increased concentration of IL-8 has been found in the vitreous fluids of patients with retinal neovascularization (Yoshida 1998). In the angiogenic process, IL-8 does not only act as a chemoattractant for neutrophils, but also as an autocrine and paracrine stimulus for endothelial cell proliferation and capillary tube formation in vitro. Beside IL-8, also other pro-inflammatory cytokines like IL-6, IL-2 and TNF-alpha have been attributed a role in pathological angiogenesis. A role of IL-6 in ocular neovascularization is supported by the finding, that IL-6 was significantly increased in the aqueous humor of neovascular glaucomas (Chen 1999).

Fluorescein angiography has been established as the major diagnostic tool for the assessment of retinal vessel conditions in ocular diseases. In this method, the fluorescein dye is injected intravenously, then it is excited around 490 nm and the emitted light of 520-530 nm is detected by a fundus camera or scanning laser opthalmoscope (SLO). Due to a fast distribution of the dye in the body and due to a subsequently fast dilution of the dye, the time frame for detecting fluorescence in the eye with a good contrast is very limited, usually only a few minutes. In this method, the hyperfluorescence detected in the tissue is simply indicative for dye leakage. This rather unspecific leaking effect—usually facilitated by neovascular damage—can hinder a determination of the very fine vascular sprouts, being characteristic for angiogenic tissue. No information on the degree of angiogenesis or on the so-called angiogenic potential of the vasculature can be gained. It is another drawback of the method that fluorescence intensity is strongly decreased by increase retinal pigment or subretinal blood of hemorrhages (Zeimer et al. U.S. Pat. No. 6,440,950).

The latter has been partially overcome by the application of the infrared dye indocyanine green (ICG) since the longer wavelengths have better penetration properties through retinal pigment and hemorrhages (Ebrahim et al., 2005). However, ICG extravasates less than fluorescein since it is strongly protein bound, so the distribution kinetics of ICG and fluorescein are not directly comparable.

To overcome the shortcomings of a short time frame due to fast dye dilution and leakage into the vasculature, fluorescence dyes were encapsulated in neutral liposomes which lead to prolonged circulation times of the dye (Peyman et al., 1996). However, the liposomally encapsulated dyes did not extravasate at sites of inflammation or neovascularization, thus no information on these critical properties were provided. Subsequently, a combination of free and encapsulated dyes was assessed (Peyman et al., 1996).

In another approach to improve the diagnostic methods, a fluorescent dye was encapsulated in temperature sensitive liposomes at a quenching concentration and administered intravenously. The dye was released from the liposomes by heating the vessel of interest by a laser. One drawback of this method is the use of laser power above the permitted non-damaging threshold (Peyman et al., 1996).

Based on the angiographic assessment, CNV can be classed into Classic CNV, with a defined hyperfluorescence of more than 50% of all lesions in the early phase, and Occult CNV with no or only strippled hyperfluorescence in the early phase and hyperfluorescence at a later time point. Classification of CNV is considered when an appropriate treatment of CNV is selected. In general the angiographic methods in use today only allow detection of rather late events in the CNV. They also do not provide information on a cellular level, like the angiogenic activation of choroidal or retinal endothelial tissue. Until recently, thermal laser photocoagulation has been the only well-established treatment modality for CNV in wet AMD. The laser is absorbed in the RPE and induces coagulation in the underlying choroidal vessels, thereby leading to the destruction of choroidal neovasculature. However, in patients with subfoveal CNV, laser photocoagulation can not be performed. Also, this treatment is only beneficial for relatively small-sized CNV, because the photocoagulation destroys the viable neurosensory retina overlying the treated CNV. As the treatment is restricted to Classical extrafoveal CNV, only less than about 10% of patients are eligible for the treatment.

To follow the strategy of occluding the neovascular blood vessels without the major drawback of injuring overlying tissue layers by high thermal laser energy, photodynamic therapy (PDT) was developed. This therapeutic approach employs in general the systematic administration of a photosensitizer which is activated by a non-thermal laser. The first drug approved in the US for the use in wet AMD was a liposomal formulation of a benzoporphyrin derivative (BPD), for the use in PDT. As disclosed in EP 1609465 by Kataoka et al., the photosensitizer can also be encapsulated into polymer micelles. Upon activation of the photosensitizer, oxygen radicals are generated that induce apoptosis in endothelial cells of the neovasculature, thereby occluding the vessel. Benzoporphyrin derivative also exert a cytotoxic effect per se (Bressler and Bressler, 2000) (Ebrahim et al., 2005). In the liposomal formulation, a targeting of BPD to the LDL receptor, which is highly expressed in angiogenic tissue, has been described. Although a reduction of the risk of loss of vision underlines the clinical benefit of PDT, an improvement of vision is not frequently observed. Several compounds, formulations, methods of manufacture and applications are described is U.S. Pat. No. 4,883,790; U.S. Pat. No. 4,920,143; U.S. Pat. No. 5,095,030; U.S. Pat. No. 5,214,036; U.S. Pat. No. 5,707,608; U.S. Pat. No. 5,798,349; U.S. Pat. No. 6,078,666 and US 2005/0152960.

The increasing understanding of angiogenic processes that are involved in ocular neovascularization (Campochiaro, 2000) (Adamis et al., 1999) has lead to development of new therapeutic strategies. Most of these new therapies target the vascular endothelial growth factor (VEGF) pathway that has been shown to play a central role in retinal as well as choroidal neovascularization. The first drug targeting the VEGF-A pathway that was approved for the treatment of wet AMD was a pegylated anti VEGF aptamer (pegaptanib, Macugen™) that inhibits VEGF165. A recombinant humanized anti-VEGF monoclonal antibody fragment (ranibizumab, Lucentis™) is currently evaluated in late stage clinical development. Other agents that are currently evaluated are a monoclonal recombinant humanized antibody against VEGF (bevacizumab, Avastin™) a receptor-immunglogulin fusion protein (VEGF-TRAP), a VEGF receptor analogue (sFLT 1), inhibitors of receptor tyrosine kinase or protein kinase C and siRNAs interfering with VEGF RNA (van Wijngaarden et al., 2005).

A major drawback of the aptamer and antibodies is their current route of administration by repeated intravitreal injection, which is inconvenient for the patient, expensive for the health care system (sterile operation room etc.) and poses the risk of ocular infections, vitreous hemorrhage, retinal detachment, and lenticular trauma (Ebrahim et al., 2005). The frequent use of anti VEGF antibodies in the treatment of cancer has also brought up concerns on the safety of anti VEGF therapy (Ratner, 2004), but this issue might be addressed by low dosing or targeted delivery of the compounds.

Although intravitreal injection is related to the above mentioned risks that represent a possible limitation of the clinical utility, many drugs destined for the posterior segment of the eye, like the retina or the choroid, are administered by the intraocular route to achieve drug levels at a therapeutical concentration. The systemic delivery of drugs to the posterior segment of the eye is limited by the blood retinal barrier (Ebrahim et al., 2005) (Olejnik and P., 2005). Thus, to achieve therapeutic concentrations of systemically administered drugs, these drugs have to be dosed at very high levels, resulting in unwanted side effects, as most of the drugs used for the treatment of ocular neovascularization do not have a highly selective mode of action. Since wet AMD is not a life-threatening indication, a balance of side effects and therapeutic success has to be found.

Topical administration of drugs to a posterior site of faces even bigger hurdles due to poor corneal absorption, rapid precorneal elimination, rapid anterior segment elimination and large diffusional path length in the eye (Olejnik and P., 2005).

Sustained release formulations or implantable depot devices have been developed to decrease the frequency of invasive treatment of the eye (Ebrahim et al., 2005) (Moshfeghi and Peyman, 2005) (Yasukawa et al., 2005). These formulation usually comprise liposomes or polymeric microcapsules/particles. Liposomal formulations have also been evaluated for the delivery of drugs by topical administration. It has been found, that the corneal uptake of such nanoparticulate formulations is increased when the particles are positively charged (Rabinovich-Guilatt et al., 2004). Cationic lipid formulations for topical administration for the treatment of ocular disorders are also disclosed in US 2004/0224010 by Hofland et al. Emulsions comprising positively charged lipidic nanoparticles for topical administration or for intra- or periocular injection are described by Benita et al in WO 03/053405 and De Kosak et al. in WO 03/053405.

To increase the drug concentration at the target site for the treatment of choroidal neovascularization by systemic administration, passive and active targeting of drugs has been suggested (Kimura et al., 2001). It is assumed that a passive targeting of drugs to neovasculature and surrounding tissue can be achieved by conjugation of the drugs to a water-soluble polymer, based on the enhanced permeation and retention effect. Active targeting to CNV could be achieved by conjugation of drugs to antibodies that are specific to the endothelium of CNV, but have no cross-reactivity to normal tissue. Unfortunately no such antibody has been clinically evaluated yet.

Although the molecular understanding of the pathogenic mechanisms of ocular neovascularization diseases has grown tremendously, the diagnostic and therapeutic options are still very limited.

As discussed above, the currently applied diagnostic methods detect the neovascularization in a late stage, when tissue destruction has already taken place, only allowing the diagnosis in late stage. The diagnosis of occult CNV is very difficult with current methods and prone to misinterpretation. A proper determination of the degree of neovascularization or the detection of angiogenesis as the key driver of the neovascular process on the cellular level is not empowered by the current methods. The possibility of the detection of angiogenic processes in the eye would allow an improved application of the new therapeutic strategies which are based on anti-angiogentic intervention. In general, an earlier detection of neovascularization and a more differentiated analysis of the disease would improve selectivity and schedule of therapeutic intervention and clinical result.

To improve the clinical outcome of the treatment of ocular neovascularization diseases like age related macular degeneration (AMD) or angioproliferative retinopathy (e.g., DR), new treatment options are needed and/or current therapies have to be improved. One of the biggest problems of the current therapies is the delivery of the drug, as intravitreous injection is related to major drawbacks and systemic administration of drugs does not reach a therapeutic level or might cause undesired side effects.

### DESCRIPTION OF THE INVENTION

It was the underlying problem of the present invention to improve the current diagnosis and therapy of ocular neovascularization diseases.

The problem underlying is solved by the invention as disclosed herein and in its embodiments.

A first embodiment of the present invention relates to a method of selectively delivering at least one active agent to the angiogenic sites of neovascular ocular endothelium, comprising the use of a systemically administered cationic colloidal carrier preparation comprising at least one active agent. Preferably, the cationic colloidal carrier is a liposome. Further, the cationic colloidal carrier preparation preferably has a positive zeta potential.

Depending on the purpose of the method, the active agent may be either a therapeutic agent or a diagnostic agent. The composition may also comprise more than one therapeutic agent, or more than one diagnostic agent, or a combination of a therapeutic agent and a diagnostic agent.

In a mouse model for laser induced choroidal neovascularization, a model that resembles the neovascularization of macular degeneration, it was surprisingly found, that Oregon Green, ICG, or fluorescein encapsulated in systemically administered liposomes comprising a positive zeta potential showed a sustained staining of the angiogenic choroidal neovascuate over approximately 80-240 min, indicating an accumulation of the cationic liposomes at these angiogenic neovascular sites. Accumulation of liposomes comprising ICG at angiogenic neovascular sites could even be observed for about 24 h. The structure of the new vessels was well resolved. Hardly any background fluorescence could be observed demonstrating the specificity of the effect. Only a very weak and transient binding to the resting retinal vasculature could be observed shortly after the administration of the liposomes.

In contrast to the results observed for cationic liposomes, the systemic application of Oregon green in neutral liposomes resulted in a transient but intense staining of the resting retinal vasculature. No accumulation of the neovascular sites could be observed. The comparison of neutral and cationic liposomes elucidates the specificity of the targeting effect of the cationic liposomes to the angiogenic neovascular sites.

When the free fluorescent dye was administered at equivalent concentrations, no increased staining of the vasculature, especially not of the neovasculature could be observed after 60 min. In comparison to the cationic liposomes, this demonstrates a clear concentration of the administered agent at the angiogenic sites.

These observations demonstrate a selective delivery of a cationic colloidal carrier preparation comprising an active agent to the angiogenic sites of neovascular ocular endothelium after its systemic administration as the comprised agent is specifically accumulated to an elevated concentration at these sites. In a preferred embodiment the cationic colloidal carrier preparation is a liposome.

Therefore it is a further embodiment of the present invention to use a cationic colloidal preparation comprising at least one active agent for the manufacture of a pharmaceutical composition for the diagnosis of an ocular neovascularization disease whereas such compositions are systemically administered. Especially for the diagnosis of choroidal neovascularization, e.g. for the diagnosis of macular degeneration, near infrared fluorescent dyes are particularly advantageous, because the longer IR wavelengths have better penetration properties through retinal pigment and hemorrhages compared to visible wavelength. Accordingly, it also an aspect of the invention to disclose a composition comprising a cationic colloidal carrier preparation comprising a near-infrared fluorescent dye. Fluorescein is also an advantageous dye in the context of the invention, since diagnostic equipment to detect fluorescein is already broadly established. Accordingly, in another preferred embodiment of the present invention, the inventive composition comprises fluorescein or a derivative as dye. Preferably, the preparation comprises a positive zeta potential, and also preferably, it comprises liposomes.

Current assessment of CNV by fluorescein angiography is based on pathological changes within the choroid, the RPE, and the vasculature, as for example vascular leakage (Holz et al. Age-related macular degeneration (2004), 93-94) that occur during the progression of the disease. It could be shown that binding of cationic liposomes in the choroid is indicative of activation and/or inflammation and/or a change of the surface properties of endothelial cells and/or proliferation in the choroid which are rather early events in the course of the disease. Thus, cationic liposomes comprising diagnostically active agents might facilitate an earlier diagnosis as compared with methods of the prior art. Cationic liposomal diagnostics might be especially useful in “high risk” patients that already suffer from dry AMD or in cases in which wet AMD has already been diagnosed in the fellow eye. These patients could be examined using the inventive diagnostic compositions in intervals of 3-12 month. Furthermore such an early detection of disease progression might help to improve the treatment schedule, as for example anti-VEGF based therapies.

The concept of the use of a systemically administered cationic colloidal carrier preparation comprising of an active agent for the selective delivery of said active agent to the angiogenic sites of neovascular ocular endothelium is not only restricted to diagnostic applications, but can also be employed in a therapeutical application.

It is another important aspect of the invention to disclose the use of a cationic colloidal preparation comprising at least one active agent for the manufacture of a pharmaceutical composition for the prevention and/or treatment of an ocular neovascularization disease wherein said composition is administered systemically. At least one active agent is a therapeutic agent. Also, a therapeutic and a diagnostic agent may be comprised in the composition. Preferably, the therapeutic agent is an antiangiogenic agent.

As described above, a variety of antiangiogenic agents are used or evaluated in the therapy of ocular neovascular diseases, especially for the treatment of AMD. All these agents might be used as an active agent within the context of the current invention. Another preferred active agent are photosensitizers, especially porphyrin or derivatives or precursors thereof for the use in a photodynamic therapy.

Within the context of a therapeutic application of the invention, a method of treating or preventing an ocular neovascular disease comprising the systemic administration of a cationic colloidal preparation comprising at least one active agent is disclosed herein.

Further, it was surprisingly found that cationic colloidal carriers comprising a therapeutic agent inhibit the release of the pro-inflammatory cytokine IL-6 and the chemokine IL-8 in human vascular endothelial cells stimulated by TNFα. Even more surprisingly also cationic colloidal carriers comprising no further therapeutic agent inhibited the release of the pro-inflammatory cytokines. As described afore, the inflammatory process and the action of pro-inflammatory cytokines, especially IL-8 and IL-6, are considered to promote ocular neovascularization.

The anti-inflammatory effect of the cationic colloidal carriers is also observed in vivo. In a Carrageenan-induced paw inflammation model, cationic colloidal carriers comprising a therapeutic agent reduced inflammation, as embodied by paw swelling. The effect could also be observed for cationic colloidal carriers comprising no further therapeutic agent.

Thus, a further embodiment of the invention refers to a method for reducing the release of pro-inflammatory cytokines in the course of an ocular neovascularization disease, comprising the administration of a cationic colloidal carrier preparation, which preferably comprises a therapeutic agent. Preferably, the pro-inflammatory cytokines are IL-6 and/or IL-8. Preferably, the cationic colloidal carrier is a liposome. More preferably, the cationic colloidal carrier comprises a positive zeta potential.

Still a further embodiment of the invention refers to a method of reducing inflammation, preferably in the course of an ocular neovascularization disease, comprising the administration of a cationic colloidal carrier. The invention also refers to the use of a cationic colloidal carrier for the manufacture of a medicament for the treatment of inflammation in the course of an ocular neovascularization disease. Preferably, the cationic colloidal carrier comprises a therapeutic agent. Preferably, the cationic colloidal carrier is a liposome. More preferably, the cationic colloidal carrier comprises a positive zeta potential.

The targeting of cationic liposomes to angiogenic endothelial cells is disclosed in WO 98/40052 by McDonald et al. WO 01/82899 by Schulze et al. suggest the use of cationic nanoparticles in the context of the treatment of retinopathy. None of the disclosures suggest the selective delivery of an active agent to the angiogenic sites of neovascular ocular endothelium by the systemic administration of a cationic colloidal carrier preparation, or the use of such systemically administered preparation for the therapy or diagnosis of ocular neovascularization diseases, especially of AMD. Both applications explicitly teach the targeting of cationic liposomes/carriers to the endothelium of an angiogenic tumor tissue or a an inflamed pulmonal tissue. It could not be predicted from these documents that the overall properties of angiogenic endothelial cells of ocular neovasculature also facilitate the targeting of said cationic nanoparticles. Especially it could not be predicted that the targeting of a systemically administered preparations allows the accumulation of the comprised active agent in a magnitude that allows a therapeutic or diagnostic application.

The embodiments of the present invention present new means for the treatment and/or diagnosis of ocular neovascularization diseases with several advantageous properties:
Active agents for the treatment and/or diagnosis of ocular neovascularization diseases can be delivered in a targeted mechanism.
The angioproliferative potential can be diagnosed in an early state, consequently a specific treatment can be started earlier, leading to an improvement of the clinical outcome. For example, up to date wet AMD is usually diagnosed in a late stage when highly sensitive tissue is already destroyed. Thus, the present invention allows an early diagnosis of ocular neovascularization diseases.
The degree of neovascularization and angiogenic potential can be determined on a cellular level.
Diagnosis of occult CNV is improved in wet AMD.
Diagnosis allows the determination of angiogenesis or the activated state of choroidal blood vessels, thereby enabling an early selection of a therapy based on an antiangiogenic concept.
Biological agents that are rather cost intensive in their production can be applied in lower amounts if the dosing regimen can be optimised based on improved diagnostic.
Based on evaluation of cationic colloidal targeting in wet AMD, the success of therapies that utilize cationic colloidal carriers can be predicted.
With dyes in the green fluorescence range such as Oregon Green 488 or fluorescein, the existing instrumentation (Heidelberg Retinograph) can be used without modifications. Similarly, with liposomal dyes such as ICG or Alexa Fluor, the existing instrumentation can be used.
The ratio of therapeutic effect of a drug in relation to the undesired side effects of an active agent is improved.
Administration by intravitreal injection might be avoided, consequently also avoiding the related major side effects.
By applying cationic colloidal targeting in PDT (e.g., by encapsulation of a photosensitizer in cationic liposomes), the success of PDT can be enhanced due to improved specificity of the photosensitizer.
The transition of dry AMD to wet AMD can be diagnostically monitored.

Detailed embodiments of the invention related to the disclosed cationic colloidal carrier preparations, pharmaceutical compositions and other aspects of the invention related to the treatment and diagnosis of ocular neovascularization are described in the following specifications and examples.

“About” in the context of amount values refers to an average deviation of maximum +/−20%, preferably +/−10% based on the indicated value. For example, an amount of about 30 mol % cationic lipid refers to 30 mol %+/−6 mol % and preferably 30 mol %+/−3 mol % cationic lipid with respect to the total lipid/amphiphile molarity.

“Active agent” or “active compound” refers to an agent or compound that is diagnostically or therapeutically effective.

“Amphiphile” refers to a molecule, which consists of a water-soluble (hydrophilic) and an oil-soluble (lipophilic) part. The lipophilic part preferably contains at least one alkyl chain having at least 10, preferably at least 12 carbon atoms.

“Angiogenic” refers to cells or tissue being in the process of angiogenesis. Angiogenesis is the formation of new blood vessels from preexisting vessels. Angiogenesis occurs in different processes, for example neovascularization, where endothelial, vasculogenesis, where the vessels arise from precursor cells de novo; or vascular expansion, where existing small vessels enlarge in diameter to form larger vessels. Angiogenic cells are proliferating at a rate substantially higher than their normal proliferation rate in general.

“Angiogenic potential” (or angioproliferative potential) refers to the capability of endothelial cells to undergo angiogenesis. It indicates the activation of the cells to undergo angiogenesis

“Antiangiogenic” refers to a mechanism (e.g., drug mechanism of action) which interferes with the angiogenic pathway.

“Carrier” refers to a diluent, adjuvant, excipient, or vehicle which is suitable for administering a diagnostic or therapeutic agent. The term also refers to a pharmaceutical acceptable component(s) that contain(s), complex(es) or is/are otherwise associated with an agent to facilitate the transport of such an agent to its intended target site. Carriers include those known in the art, such as liposomes, polymers, lipid complexes, serum albumin, antibodies, cyclodextrins and dextrans, chelate, or other supramolecular assemblies.

“Cationic” refers to an agent that has a net positive charge or positive zeta potential under the respective environmental conditions. In the present invention, it is referred to environments where the pH is in the range between 3 and 9, preferably between 5 and 8, especially between 7 and 8.

“Colloidal” refers to matter in the size range between about 1 nm and about 5000 nm. For example, the colloidal matter can be a liposome, a solid lipid particle, a micelle, a solid drug particle, a polymer or polymer particle, a solid gold or metal particle, a quantum dot, a dendrimer, a fullerene, a carbon nanotube, a (polymer) capsule, supramolecular assemblies, or any other nanoparticle. Preferably, the colloidal carrier is a liposome.

“Cryoprotectant” refers to a substance that helps to protect a species from the effect of freezing.

“Derivative” refers to a compound derived from some other compound while maintaining its general structural features. Derivatives may be obtained for example by chemical functionalization or derivatization.

“Drug” as used herein refers to a pharmaceutically acceptable pharmacologically active substance, physiologically active substances and/or substances for diagnosis use.

“Diagnostic agent” or “diagnostically active agent” refers to a pharmaceutical acceptable agent that can be used to localize or visualize a target region by various methods of detection. Such agents include those known in the art, such as dyes, fluorescent dyes, infrared dyes, gold particles, iron oxide particles and other contrast agents including paramagnetic molecules, X-ray attenuating compounds (for CT and X-ray) contrast agents for ultrasound, magnetic resonance imaging (MRI), X-ray emitting isotopes (scintigraphy), and positron-emitting isotopes (PET).

“Lipid” refers to its conventional sense as a generic term encompassing fats, lipids, alcohol-ethersoluble constituents of protoplasm, which are insoluble in water. Lipids are composed of fats, fatty oils, essential oils, waxes, steroid, sterols, phospholipids, glycolipids, sulpholipids, aminolipids, chromolipids, and fatty acids. The term encompasses both naturally occurring and synthetic lipids. Preferred lipids in connection with the present invention are: steroids and sterol, particularly cholesterol, phospholipids, including phosphatidyl and phosphatidylcholines and phosphatidylethanolamines, and sphingomyelins. Where there are fatty acids, they could be about 12-24 carbon chains in length, containing up to 6 double bonds. The fatty acids are linked to the backbone, which may be derived from glycerol. The fatty acids within one lipid can be different (asymmetric), or there may be only 1 fatty acid chain present, e.g., lysolecithins. Mixed formulations are also possible, particularly when the non-cationic lipids are derived from natural sources, such as lecithins (phosphatidylcholines) purified from egg yolk, bovine heart, brain, or liver, or soybean.

“Liposome” refers to a microscopic spherical membrane-enclosed vesicle (about 50-2000 nm diameter) made artificially in the laboratory. The term “liposome” encompasses any compartment enclosed by a lipid bilayer. Liposomes are also referred to as lipid vesicles. In order to form a liposome the lipid molecules comprise elongated nonpolar (hydrophobic) portions and polar (hydrophilic) portions.

The hydrophobic and hydrophilic portions of the molecule are preferably positioned at two ends of an elongated molecular structure. When such lipids are dispersed in water they spontaneously form bilayer membranes referred to as lamellae. The lamellae are composed of two mono layer sheets of lipid molecules with their non-polar (hydrophobic) surfaces facing each other and their polar (hydrophilic) surfaces facing the aqueous medium. The membranes formed by the lipids enclose a portion of the aqueous phase in a manner similar to that of a cell membrane enclosing the contents of a cell. Thus, the bilayer of a liposome has similarities to a cell membrane without the protein components present in a cell membrane.

As used in connection with the present invention, the term liposome includes multilamellar liposomes, which generally have a diameter in the range of about 1 to about 10 micrometers and are comprised of anywhere from two to hundreds of concentric lipid bilayer alternating with layers of an aqueous phase, and also includes unilamellar vesicles which are comprised of a single lipid bilayer. The latter can be produced by subjecting multilamellar liposomes to ultrasound, by extrusion under pressure through membranes having pores of defined size, or by high pressure homogenization. A further result of these procedures is, that often well defined size distributions of the liposomes are achieved. By extrusion through membranes of defined pore size (typical values are 100, 200, 400 or 800 nm), liposomes with a size distribution close to the pore size of the membrane can be achieved. By ultrasound and high pressure homogenisation procedures, defined size distributions are obtained by molecular self-organization as a function of the experimental conditions.

“Liposomal preparation” and “liposomes” are used synonymously throughout the present application. The liposomal preparation may be a component of a “pharmaceutical composition” and may be administered together with physiologically acceptable excipients such as a buffer.

“Lysolipid” refers to a lipid where one fatty acid ester has been cleaved resulting in a glycerol backbone bearing one free hydroxyl group.

“Lysophospholipid” refers to a phospholipid where one fatty acid ester has been cleaved resulting in a glycerol backbone bearing one free hydroxyl group.

“Macula” is the central area of the retina, responsible for vision, necessary for reading.

“Macular degeneration” or “AMD” refers to a disease of the central retina (macula). There are early and late stages of which the latter can be divided in late dry and late wet AMD. Wet AMD is associated with choroidal neovascularization.

“Membrane bound active agent” refers to an active compound or drug which will—based on its physicochemical characteristics—associate with the membrane of a liposome or with the lipid phase of the carrier (e.g., due to its lipophilicity or due to its charge).

“Mol percent” or “mol %” refers to the molar ratio, given in percent, of lipid molecules and active agent molecules that constitute a liposome. Thus, e.g., a liposomal composition which comprises 5 mol DOTAP, 4,7 mol DOPC, and 0,3 mol paclitaxel comprises 50 mol % DOTAP, 47 mol % DOPC, and 3 mol % paclitaxel.

“Negatively charged lipids” refer to lipids that have a negative net charge in an environment where the pH is in the range between 3 and 9, preferably between 5 and 8, especially between 7 and 8.

“Neovascularization” refers to the new growth blood vessels, especially to the pathologic growth of blood vessels.

“Nonmembrane bound active agent” refers to an active compound or drug which will—based on its physicochemical characteristics—not associate with the membrane of a liposome or with the lipid phase of a carrier (e.g., due to its hydrophilicity).

“Ocular neovascularization diseases” refers to diseases affecting the eye that are caused by or involve neovascularization, especially choroidal or retianal neovascularization. Examples of such diseases include, but are not limited to macular degeneration, especially wet age-related macular degeneration, retinopathy, especially proliferative diabetic retinopathy, and retinopathy of prematurity.

“Photosensitizer” refers to an agent that is activated by light, e.g. a laser, to exert its desired effect.

“Paclitaxel” (which should be understood herein to include analogues, formulations, and derivatives such as, for example, docetaxel, taxotere (a formulation of docetaxel), 10-desacetyl analogs of paclitaxel and 3′N-desbenzoyl-3′N-t-butoxycarbonyl analogs of paclitaxel) may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590267), or obtained from a variety of commercial sources, including for example, Sigma Chemical Co., St. Louis, Mo. (T7402 from Taxus brevifolia; or T-1912 from Taxus yannanensis). Paclitaxel should be understood to refer to not only the common chemically available form of paclitaxel, but analogs (e.g., taxotere, as noted above) and paclitaxel conjugates (e.g., paclitaxel-PEG, paclitaxeldextran, or paclitaxel-xylose).

“Particle diameter” refers to the size of a particle. To experimentally determine particle diameters, dynamic light scattering (DLS) measurements, using Malvern Zetasizer 1000 or 3000 (Malvern, Herrenberg, Germany) were performed. For quantitative data analysis (determination of Z (average and PI), in all cases parameters (refractive index, density, viscosity) of pure water were plugged in, even if the aqueous phase contained cryoprotectants to a certain extend. Therefore, for the absolute numbers, a certain systematic deviation with respect to literature data may have to be taken into account.

“Pegylated lipid” refers to a lipid bearing one or more polyethylene glycol residues.

“Pharmaceutical composition” refers to a combination of two or more different materials with suitable properties for a pharmaceutical application.

“Phospholipid” refers to a lipid consisting of a glycerol backbone, a phosphate group and one or more fatty acids which are bound to the glycerol backbone by ester bonds.

“Positively charged lipids” refer to a synonym for cationic lipids (for definition see definition of “cationic lipids”).

“Reducing the release of pro-inflammatory cytokines” refers to a reduction of the release of at least one inflammatory cytokine, preferably by endothelial cells, of at least 25%, preferably at least 40%. Inhibition of cytokine release may be determined by a cell culture assay, using TNFα stimulated endothelial cells as described in Example 15.

“Retinopathy” refers to a disease of the retina which can occur in association with diabetic retinopathy, vessel occlusion or retinopathy of prematurity, choroidal neovascularization or AMD.

“Sterol” refers to a steroid alcohol. Steroids are derived from the compound called cyclopentanoperhydrophenanthrene. Well-known examples of sterols include cholesterol, lanosterol, and phytosterol.

“Taxane” as used herein refers to the class of antineoplastic agents having a mechanism of microtubule action and having a structure that includes the unusual taxane ring structure and a stereospecific side chain that is required for cytostatic activity. Also included within the term “taxane” are a variety of known derivatives, including both hydrophilic derivatives, and hydrophobic derivatives. Taxane derivatives include, but are not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869.

“Therapeutic agent” refers to a species of agents that reduces the extent of the pathology of a disease such as an ocular neovascularization disease.

“Total lipid” refers to the amount of lipid present in a preparation. The total lipid includes all lipid that is present in the preparation. In a liposomal preparation, this lipid constitutes the membrane.

“Total liposomal components” refers to the components that constitute the liposomes. Within the context of this invention the liposome is constituted by the components that constitute the membrane and the active agent comprised in the liposome.

“Treatment”, “treating”, “treat” and the like are used herein to generally mean obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

“Zeta potential” refers to measured electrical potential of a colloidal particle in aqueous environment, measured with an instrument such as a Zetasizer 3000 (Malvern Instruments) using Laser Doppler micro-electrophoresis under the conditions specified. The zeta potential describes the potential at the boundary between bulk solution and the region of hydrodynamic shear or diffuse layer. The term is synonymous with “electrokinetic potential” because it is the potential of the particles which acts outwardly and is responsible for the particle's electrokinetic behaviour.

It is a general aspect of the present invention, that the therapeutic agent can be an organic or anorganic small molecule, a polypeptide agent like a protein, an antibody (monoclonal or polyclonal) or an antibody fragment, a fusion protein, a short peptide, or an oligo- or polynucleotide like an oligoaptamer, aptamer, a gene fragment, plasmids, ribozyme, small interference RNA (siRNA), nucleic acid fragment, etc.

In one embodiment of the present invention, the therapeutic agent is an antiangiogenic agent. Preferably, said antiangiogenic agent is a cytotoxic or cytostatic agent, preferably a antineoplastic agent especially antimitotic agent like a taxane, an anthracyclin preferably doxorubicin or epirubicin, a statin, a depsipeptide, thalidomide, other agents interacting with microtubuli such as discodermolide, laulimalide, isolaulimalide, eleutherobin, epothilone, Sarcodictyin A and B, antimetabolites preferably antifolates, preferably methotrexate, alkylating agents especially platinum containing compounds like cisplatin, carboplatin, DNA topoisomerase inhibiting agents, preferably camptothecin, RNA/DNA antimetabolites, especially 5-fluorouracil, gemcitabine or capecitabine. The antiangiogenic agent can also be a protease inhibitor, preferably an inhibitor of plasminogen, urokinase like plasminogen activator (uPA) or an matrix metalloproteinases (MMPs), especially an inhibitor of MMP-1, MMP-2, MMP-3, MMP-7, MMP-8, MMP-9, MMP-10, MMP-11 or MMP-13.

In a preferred embodiment of the present invention, the taxane is docetaxel or paclitaxel or a derivative thereof. The cationic colloidal carrier preparation may comprise paclitaxel in an amount of at least about 2 mole % to about 8 mole %, preferably from at least 2.5 mole % to about 3.5 mole %.

In another preferred embodiment, the preparation may comprise the paclitaxel derivative succinyl-paclitaxel (WO 2004/002455) in an amount of up to 15 mol %, more preferably about 10-12 mol %.

In a more preferred embodiment the cationic liposomal preparation comprises DOTAP, DOPC and paclitaxel in a ratio of about 50:47:3. This formulation is also designated MBT-0206 or EndoTAG™-1. EndoTAG™-1 has a lipid content of 10 mM in a 10% m/m trehalose dihydrate solution. The manufacture of such a formulation is disclosed in WO 2004/002468.

It is another aspect of the current invention, that the antiangiogenic agent is an antagonist of a growth factor like VEGF, PDGF, EGF, FGF, preferably an antagonist of VEGF. The antagonist of VEGF may be an antibody or antibody fragment like bevacizumab or rhufab V2 (ranibizumab), a soluble receptor or a fusion protein with receptor fragments like VEGF-TRAP_{R1R2}, a growth factor receptor kinase inhibitor, preferably a KDR selective receptor tyrosine kinase inhibitor like SU5416, a protein kinase C inhibitor, PTK787, a nucleic acid based antagonist like siRNAs against VEGF or VEGFR1 or 2, or an aptamer like pegaptanib sodium, or squalamine.

The therapeutic agent of the invention may also be an anti-inflammatory agent such as synthetic glucorticoids, mineralocorticoids, hydrocortisone, dexamethasone, fluocinolone, prednisone, prednisolone, methylprednisolone, fluorometholone, betamethasone and triamcinolone, squalamine, anecortave acetate, a non-steroidal anti-inflammatory agent such as salicylate, indomethacin, ibuprofen, diclofenac, flurbiprofen, piroxicam or a COX2 inhibitor. Preferably, the anti-inflammatory agent is amicolone, anecortave acetate or squalamine.

In another preferred embodiment of the invention, the therapeutic agent is an antagonist against cellular adhesion molecules, especially antibodies directed against alpha5 beta1integrin, alpha5 beta3 integrin, or alpha5 beta5 integrin or RGD peptides. In a specially preferred embodiment said antagonists are RGD peptides. The peptides can be linear or cyclic, optionally the peptides are derivatized.

The therapeutic agent can also be a cytokine like an interferon or interleukin, or a chemokine.

It is also an aspect of the current invention that the therapeutic agent can be a photosensitizer like a porphyrin photosensitizer like hematoporphyrin and derivatives thereof like dihematoporphyrin ether, tetraphenyl porphyrins, tetraethylporphyrins, tetrapyridyl porphyrins, protporphyrin IX, phtalocyanine and derivatives like Zn(II)-phthalocyanine, Ge(IV)-phthalocyanine, Zn(II)-2,3naphthalocyanine and Si(IV)-naphthalocyanine green porphyrins, ternoporfin and talaporfin, chlorines like chlorin e6 trimethyl ester and pheophorbide, purpurin, texaphyrin and derivatives, tin ethyletiopurpurium (SnET₂), ATX-S10, MV6401. Preferred are hydro-mono benzoporphryins and SnET₂. Some suitable porphyrins are disclosed in (U.S. Pat. No. 4,883,790; U.S. Pat. No. 4,920,143; U.S. Pat. No. 5,095,030; U.S. Pat. No. 5,171,749). Further, the photosensitizer may be porphyrin precursor such as 5-aminolevulinic acid (ALA) or a salt or derivative such as an ester or amide thereof. The disclosed preparations comprising a photosensitizer are applied in a photodynamic therapy wherein the photosensitizer is activated by light. The preparation comprising a photosensitizer can also comprise an diagnostic agent, allowing the detection of neovascular sites and the subsequent, specific occlusion of the neovascular vessels.

In another embodiment of the invention, the active agent is a diagnostically active agent. The diagnostically active agent is selected from a group comprising fluorescent labels, histochemical labels, immunohistochemical labels, radioactive labels, especially metal ions or metal ion chelates (preferably chelates from transition metals such as gadolinium, lutetium, or europium) used as contrast agents for MRI, CT and X-ray. Other preferred labels are radioisotopes, preferably isotopes of Iodine, Indium, Gallium, Ruthenium, Mercury, Rhenium, Tellurium, Thulium, and more preferably Technetium.

In a more preferred embodiment, the fluorescent label is a fluorescence dye in the visual and near-infrared wavelength range, preferably fluorescein and derivatives like 6-carboxy-fluorescein, Oregon Green and derivatives, Pacific Blue, Rhodamine especially Lissamine Rhodamine, Alexa Fluor 790, or a cyano dye like indocyanine green (ICG), or 1,1′-dioctadecyltetramethylindotricarbocyanineiodide (DiR) and their derivatives. In a preferred embodiment, the dye is coupled to a lipid molecule. Preferably, the fluorescence of the these dyes will be detected by scanning laser opthalmoscopy (SLO) or by means of a fundus camera.

It is the purpose of the present invention to be used within the field of neovascularization disease in the eye, preferably for the therapy and/or diagnosis of said disease. The neovascularization disease can by caused by choroidal or retinal neovascularization. Preferred are macular degeneration such as age related macular degeneration, or retinopathy, preferably proliferative diabetic retinopathy, proliferative retinopathy after vessel occlusion, and retinopathy of prematurity.

It is also an aspect of the current invention, that the disclosed preparations are administered systemically, preferably intravenously. The preparations are administered in a therapeutically or diagnostically effective dose, which will be different for the comprised active agent, the treated/diagnosed disease, or the subject to which administration occurs. The skilled person is able to determine these doses.

The disclosed preparations comprising an active agent may be administered in form of a combination therapy with an at least second active agent which is useful in the treatment of ocular diseases such as ocular neovascularization, preferably an anti VEGF active agent.

In a preferred embodiment the preparation is administered to a human patient in need of a therapy or a diagnosis.

The cationic colloidal carrier comprised in the cationic colloidal carrier preparation disclosed herein can be a liposome, a solid lipid particle, a micelle, a solid drug particle, a polymer or polymer particle, a solid gold or metal particle, a quantum dot, a dendrimer, a fullerene, a carbon nanotube, a (polymer) capsule, or any other nanoparticle in the size range between about 1 nm and about 5000 nm. Preferably, the size of the colloidal carrier is between 10 nm and 1000 nm.

In an especially preferred embodiment the cationic carrier preparation is a liposomal preparation.

In a preferred embodiment, the colloidal carrier preparation of the present invention comprises a cationic lipid or a mixture of cationic lipids in an amount of at least about 30 mol %, more preferably at least about 50 mol % of total lipid.

The preferred cationic lipids of the liposomal preparation are N-[1-(2,3-dioleoyloxy)propyl]-N,N,N-trimethyl ammonium salts, e.g. the methylsulfate. Preferred representatives of the family of -TAP lipids are DOTAP (dioleoyl-), DMTAP (dimyristoyl-), DPTAP (dipalmitoyl-), or DSTAP (distearoyl-). Other useful lipids for the present invention may include: DDAB, dimethyldioctadecyl ammonium bromide; 1,2-diacyloxy-3-trimethylammonium propanes, (including but not limited to: dioleoyl, dimyristoyl, dilauroyl, dipalmitoyl and distearoyl; also two different acyl chains can be linked to the glycerol backbone); N-[1-(2,3-dioloyloxy)propyl]-N,N-dimethyl amine (DODAP); 1,2-diacyloxy-3-dimethylammonium propanes, (including but not limited to: dioleoyl, dimyristoyl, dilauroyl, dipalmitoyl and distearoyl; also two different acyl chain can be linked to the glycerol backbone); N-[1-(2,3-dioleyloxy)propyl]-N,N,N-trimethylammonium chloride (DOTMA); 1,2-dialkyloxy-3-dimethylammonium propanes, (including but not limited to: dioleyl, dimyristyl, dilauryl, dipalmityl and distearyl; also two different alkyl chain can be linked to the glycerol backbone); dioctadecylamidoglycylspermine (DOGS); 3β[N—(N′,N′-dimethylamino-ethane)carbamoyl]cholesterol (DC-Chol); 2,3-dioleoyloxy-N-(2 (sperminecarboxamido)-ethyl)-N,N-dimethyl-1-propanaminium trifluoro-acetate (DOSPA); β-alanyl cholesterol; cetyl trimethyl ammonium bromide (CTAB); diC14-amidine; N-tert-butyl-N′-tetradecyl-3-tetradecylamino-propionamidine; 14Dea2; N-(alpha-trimethylammonioacetyl)didodecyl-D-glutamate chloride (TMAG); O,O′-ditetradecanoyl-N-(trimethylammonio-acetyl)diethanolamine chloride; 1,3-dioleoyloxy-2-(6-carboxy-spermyl)-propylamide (DOSPER); N,N,N′,N′-tetramethyl-N,N′-bis(2-hydroxylethyl)-2,3-dioleoyloxy-1,4-butanediammonium iodide; 1-[2-(acyloxy)ethyl]2-alkyl (alkenyl)-3-(2-hydroxyethyl)-imidazolinium chloride derivatives as described by Solodin et al. (Solodin et al., 1995), such as 1-[2-(9(Z)-octadecenoyloxy)ethyl]-2-(8(Z)-heptadecenyl-3-(2-hydroxyethyl)imidazolinium chloride (DOTIM), 1-[2-(hexadecanoyloxy)ethyl]-2-pentadecyl-3-(2-hydroxyethyl) imidazolinium chloride (DPTIM), 2,3-dialkyloxypropyl quaternary ammonium compound derivatives, containing a hydroxyalkyl moiety on the quaternary amine, as described e.g. by Felgner et al. (Felgner et al., 1994) such as: 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimethyl-hydroxypropyl ammonium bromide (DORIE-HP), 1,2-dioleyl-oxy-propyl-3-dimethyl-hydroxybutyl ammonium bromide (DORIE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), 1,2-disteryloxypropyl-3-dimethyl-hydroxyethyl ammonium bromide (DSRIE); cationic esters of acyl carnitines as reported by Santaniello et al. (U.S. Pat. No. 5,498,633); cationic triesters of phosphatidylcholine, i.e. 1,2-diacyl-sn-glycerol-3-ethylphosphocholines. The hydrocarbon chains of the cationic lipids can be saturated or unsaturated and branched or non-branched with a chain length from C₁₂ to C₂₄. Preferably, the lipid comprises at least two hydrocarbon chains which may be different or identical.

The colloidal carrier preparation optionally comprises at least one neutral and/or anionic lipid. Neutral lipids are lipids which have a neutral net charge. Anionic lipids or amphiphiles are molecules which have a negative net charge. These can be selected from sterols or lipids such as cholesterol, phospholipids, lysolipids, lysophospholipids, sphingolipids or pegylated lipids with a neutral or negative net charge. Useful neutral and anionic lipids thereby include: phosphatidylserines, phosphatidylglycerols, phosphatidylinositols (not limited to a specific sugar), fatty acids, sterols, containing a carboxylic acid group for example, cholesterol, phosphatidylethanolamines (PE) such as 1,2-diacyl-sn-glycero-3-phosphoethanolamines including, but not limited to 1,2-dioleoylphosphoethanolamine (DOPE), 1,2-distearoylphosphoethanolamine (DSPE), or 1,2-dihexadecoylphosphoethanolamine (DHPE), phosphatidylcholines (PC) such as 1,2-diacyl-glycero-3-phosphocholines including, but not limited to 1,2-distearoylphosphocholine (DSPC), 1,2-dipalmitoylphosphocholine (DPPC), 1,2-dimyristoylphosphocholine (DMPC), egg PC or soybean PC and sphingomyelins. The fatty acids linked to the glycerol backbone are not limited to a specific length or number of double bonds. Phospholipids may also have two different fatty acids. Preferably, the further lipids are in the liquid crystalline state at room temperature and they are miscible (i.e. a uniform phase can be formed and no phase separation or domain formation occurs) with the used cationic lipid, in the ratio as they are applied. In a preferred embodiment the neutral lipid is 1,2-dioleylphosphocholine (DOPC).

The colloidal carrier preparation comprises optionally neutral and/or anionic lipids, preferably DOPC in an amount of up to about 70 mole %, preferably up to about 50 mole %, most preferably up to about 30 mole % of total lipid.

The disclosed preparations may comprise polyethylene glycol (PEG) or a derivative thereof. Preferably, the colloidal carrier preparation of the invention may comprise pegylated lipids. Pegylated lipid refers to a lipid bearing one or more polyethylene glycol residues. The lipid bearing the polyethylene glycol residue may be a anionic or cationic and preferably a neutral lipid. In a more preferred embodiment the neutral lipid is a pegylated PE and/or PC, more preferably DOPE or DSPE. Preferably, the molecular weight of PEG residues is between about 750 Da and about 5000 Da. Most preferably the pegylated lipid is DOPE pegylated with PEG₂₀₀₀. The colloidal carrier preparation of the invention may also comprise lipids which are derivatized by other biocompatible polymers that reduce non-specific interactions by steric hinderence, for example sugars like dextrans or celluloses.

The active agents of the present invention can be comprised in the colloidal carrier preparation as a derivative of said active agent coupled to a lipid component. The coupling of the active agent to a lipid compound can increase the loading efficiency and the stability of the active agent to/in the carrier, e.g. the liposome. Preferably, the agent is coupled to a neutral lipid preferably a PE such as DOPE or DHPE.

The colloidal carrier preparation may comprise a membrane bound active agent preferably in an amount of about up to 20 mol % of total liposomal components, more preferably between about 1 mol % to about 10 mol %, most preferably between about 3 mol % to about 6 mol % of total liposomal components. Alternatively or additionally, the colloidal carrier preparation may preferably comprise a nonmembrane bound active agent in an amount of about up to 50 mol % of total liposomal components, more preferably between about 1 and 30 mol % and most preferably between about 5 and 20 mol %.

The active agent may be located in the aqueous compartment of the liposome in case of a water soluble agent or bound to or integrated into the liposomal membranes in case of a insoluble/lipophilic agent. If a water soluble agent is encapsulated in the liposome, DSTAP, DPTAP or DMTAP are preferred cationic lipids to prevent a leaking of the compound from the liposome in the blood.

Within the context of the current invention, also thermolabile colloidal carriers, e.g. liposomes are disclosed. Thermolabile liposomes in general have been described by Hosokawa et al. (Hosokawa et al., 2003) and Needham et al. (Needham et al., 2000) Thermolabile liposomes are stable at 37° C., but release the comprised agent at a temperature of between about 40° C. and about 45° C. due to the transition temperature of the comprised lipids. Preferably, thermolabile liposomes comprise a fluorescent dye at a quenching concentration, or a photosensitizer, or another therapeutic agent. Release of the comprised agent can be induced by appropriate laser energy.

The colloidal carrier preparations, e.g. the liposomal preparations of the present invention can be obtained by homogenizing the hydrophobic compounds in water by a suitable method and further processing. Homogenizing can be obtained by mechanical mixing, stirring, high-pressure homogenization, adding an organic phase comprising the hydrophobic compounds to the aqueous phase, spraying techniques, supercritical fluid technology or any other technique suitable in order to obtain lipid dispersions in water.

In a preferential embodiment, the liposomal preparations of the present invention can be obtained by method like the “lipid film method” or by “ethanol injection”, which are known to those skilled in the art and are disclosed in WO 2004/002468 for example.

The colloidal carrier preparation can be dehydrated, stored for extended periods of time while dehydrated, and then rehydrated when and where it is to be used, without losing a substantial portion of its contents during the dehydration, storage and rehydration processes. To achieve the latter, one or more protective agents, such as cryoprotectants, may be present. Thus, the preparation preferably comprises a cryoprotectant, wherein the cryoprotectant can be selected from a sugar or an alcohol or a combination thereof. Preferably, the cryoprotectant is selected from trehalose, maltose, sucrose, glucose, lactose, dextran, mannitol or sorbitol. The cryprotectants are usually present in an amount of about 5% (m/v) to about 15% (m/v) with respect to the total volume of the preparation.

In a further preferred embodiment, the colloidal carrier preparation comprises trehalose in the range of about 5% (m/v) to about 15% (m/v) with respect to the total volume of the preparation.

It is one aspect of the present invention, that the cationic colloidal carrier preparation comprises a zeta potential of greater than about 20 mV, preferably greater than about 30 mV, and most preferably greater than about 40 mV when measured in about 0.05 mM KCl solution at about pH 7.5.

Preferred liposomes of the liposomal preparations disclosed in this applications are small unilamellar liposomes with an average particle diameter of about 50 nm to about 400 nm, preferably about 100 nm to about 300 nm, about 100 nm to about 200 nm.

In accordance with other aspects of the invention, the pharmaceutical composition of the invention comprises a pharmaceutically effective amount of the inventive colloidal carrier preparation together with a pharmaceutically acceptable carrier, diluent and/or adjuvant.

A further aspect of the invention refers to a composition comprising a cationic colloidal carrier preparation comprising a VEGF antagonist as an active agent.

A still further aspect of the invention refers to a composition comprising a cationic colloidal carrier preparation comprising an antagonist against cellular adhesion molecules as an active agent.

A still further aspect of the invention refers to a composition comprising a cationic colloidal carrier preparation comprising a photosensitizer as an active agent.

A still further aspect of the invention refers to a composition comprising a cationic colloidal carrier preparation comprising a siRNA molecule as an active agent. The siRNA molecule is preferably a double-stranded RNA molecule optionally comprising at least one modified nucleotide, wherein the length of the RNA strands is preferably between 19 and 25 nucleotides. Further, the siRNA molecule may comprise at least one 3′-overhang.

A still further aspect of the invention refers to a composition comprising a cationic colloidal carrier preparation comprising an aptamer as an active agent.

It should be noted that all preferred embodiments discussed for one or several aspects of the invention also relate to all other aspects. This particularly refers to the amount and type of cationic lipid, the amount and type of neutral and/or anionic lipid, the amount and type of active agent, the amount and type of further active agent for combination therapy, and the type of disorder to be treated.

The following examples should be illustrative only but are not meant to be limiting to the scope of the invention. Other generic and specific configurations will be apparent to those skilled in the art.

### FIGURE LEGENDS

FIG. 1: Fluorescence intensity of cationic (MRa0049) and neutral (MRa0050) liposomes labelled with Oregon Green. The left picture shows that in intact liposomes the fluorescence intensity in cationic liposomes (DOTAP/DOPC/DHPE-OG=60/35/5) is about twice as high as in neutral liposomes.

FIG. 2: Structure of DiR.

FIG. 3: Spectral characterization of DiR in aqueous medium.

FIG. 4: Normalized spectra of DiR in various environments. In aqueous solution (trehalose) the emission maximum of DiR is around 640 nm but shifts to about 760 nm in ethanol or when incorporated in liposomes.

FIG. 5: SLO images obtained with EndoTAG-A. Left image was obtained in IR reflection mode, middle image was taken 2 min after i.v. application, right image was taken 54 minutes after i.v. application.

FIG. 6: SLO images obtained with neutral, OG labelled liposomes. left image was obtained in IR reflection mode, middle image was taken 2 min after i.v. application, right image was taken 28 min after i.v. Application.

FIG. 7: HUVEC were incubated either with 1, 50, 100 or 500 nM EndoTAG™-1 or EndoTAG™-Placebo in EGM2 full medium (5% FBS) containing TNFα (30 U/ml) when indicated. Supernatant was harvested after 48 hrs and amount of IL-6 (left graph) and IL-8 (right graph) cytokines were measured using “BD Cytometric Bead Array”.

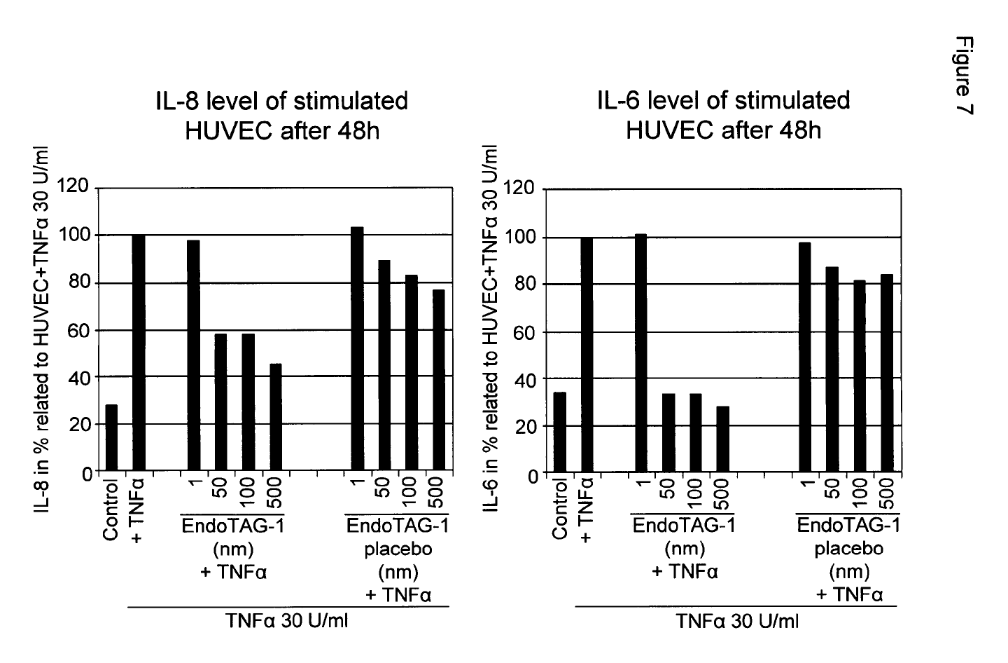

FIG. 8: The therapeutic effect of EndoTAG-1 on rat Carrageenan-induced paw inflammation was tested in Sprague Dawley rats. Shown is the mean weight difference of hind paws from 6 different animals 4 h after injection of the Carrageenan into the right hind footpad. Trehalose, EndoTAG placebo, Taxol® or EndoTAG-1 was administered iv 30 min after Carrageenan injection.

FIG. 9: EndoTAG™-1 was evaluated versus Taxol® or no treatment in a laser induced CNV animal model. EndoTAG™-1 and Taxol® were administered at a dose of 2.56 mg/kg paclitaxel. Result is shown as percentage of nonleaky lesions (score 0) in the three groups on day 10 and 17 after start of treatment.

FIG. 10: EndoTAG™-SPA and EndoTAG™-1 were evaluated versus Taxol® or no treatment in the laser induced CNV mouse model. All three therapeutic agent were administered at a dose of 0.5 mg/kg and 2.5 mg/kg. Results are shown in mean size of CNV.

### EXAMPLES

### Example 1

### Preparation of EndoTAG Labelled with Oregon Green 488

### Preparation of Liposomes

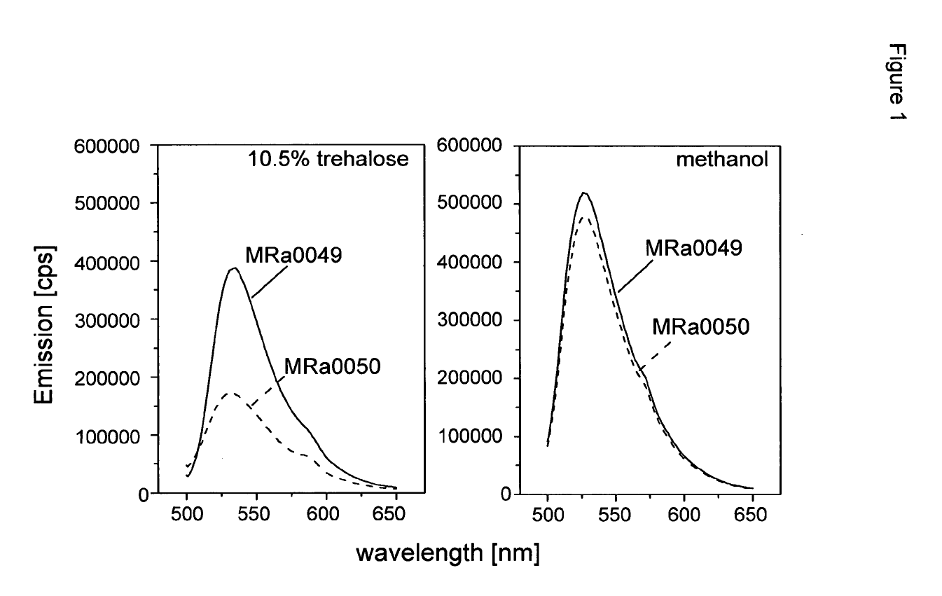

Liposomes are prepared by the lipid film method (see for example WO 2004/002468) as follows: In a round bottom flask, 0.06 mmol DOTAP, 0.035 mmol DOPC and 0.005 mmol DHPE-coupled Oregon Green (DHPE-OG) 488 501/526 nm) (Invitrogen) (are dissolved in chloroform. Next, the solvent is evaporated under vacuum and the thin lipid film is dried for about 60 min at 100 mbar. Subsequently, 10 ml of trehalose is added to the lipid film and multilamellar vesicles (MLVs) are formed spontaneously. The total liposomal component were 10 mM (total lipid content). The MLVs are extruded five times through a polycarbonate membrane with 200 nm pore size. The resulting SUVs (small unilamellar vesicles) are analysed with Photon Correlation Spectroscopy (PCS) for particle size and size distribution and with HPLC for concentration of lipid and lipid-coupled dye as described below. Fluorescence and UV/VIS spectroscopy are used to characterize the spectral properties of Oregon-Green in the liposome. First, the spectra are recorded for the free dye in methanol and compared with manufacturer's specifications. Thus, identity of the dye is assured. Next, the spectra of the dye are compared in different liposomal formulations (e.g., cationic, neutral, various ratios of DOTAP/DOPC etc) with respect to intensity and maximum. This allows (1) to determine whether the dye interacts strongly with the membrane (usually resulting in a shift of the maximum) and (2) to determine quenching phenomena. As an example, FIG. 1 shows on the left side the dye in a cationic and in a neutral liposome formulation (MRa 0049 and MRa 0050), on the right side the liposome structure has been destroyed by addition of an excess of methanol and the spectra of the dye in both formulations are identical. This illustrates how fluorescence properties of the dye are influenced by the membrane.

The formulation is stable for at least 3 months. If needed, the formulation is lyophilized.

All analytical results are within the expected range:

### 6 mM DOTAP, 3.5 mM DOPC, 0.5 mM DHPE-OG.

### PCS: Zₐᵥₑ=160 nm, PI=0.3

### Analysis of DOTAP Content by HPLC:

As stationary phase, a C8 column Luna 5μ C8 (2) 100 Å, 150×2 mm (Phenomenex) is used. The mobile phase is composed of water with 0.1% TFA (solvent A) and acetonitrile with 0.1% TFA (solvent B), the following gradient program is run:

| Time (min) | Solv. B (%) |
|---|---|
| 0.00 | 50.0 |
| 4.12 | 50.0 |
| 7.06 | 75.0 |
| 14.13 | 100.0 |
| 21.20 | 100.0 |
| 23.56 | 50.0 |
| 30.00 | 50.0 |

Column temperature: 45° C.
Injection volume: 5 μl
Wavelength for detection: 205 nm
Run time: 30 min
Retention time is 14.5 min for DOTAP, 16.5 min for DOPC and 18 min for Oregon-Green DHPE.

### Analysis of Particle Size

Particle diameters are determined by dynamic light scattering (DLS) measurements, using Malvern Zetasizer 1000 or 3000 (Malvern, Herrenberg, Germany).

### Measurement of Fluorescence Spectrum

Excitation at 490 nm, slit width±5 nm
Integration time: 0.1 s

### Example 2

### Preparation of EndoTAG Labelled with Lipid-Coupled ICG

Liposomes are prepared by the lipid film method (see for example WO 2004/002468) as follows: In a round bottom flask, 0.06 mmol DOTAP, 0.035 mmol DOPC and 0.005 mmol lipid-coupled dye ICG (790/830 nm) are dissolved in chloroform. Next, the solvent is evaporated under vacuum and the thin lipid film is dried for about 60 min at 100 mbar. Subsequently, trehalose is added to the lipid film and multilamellar vesicles (MLVs) are formed spontaneously. The MLVs are extruded (5×200 nm) and analysed with PCS for particle size and distribution and with HPLC for concentration of lipid and lipid-coupled dye as described in Example 1. Fluorescence spectroscopy is used to characterize the spectral properties of ICG in the liposome in a similar way as in example 1.

The formulation is stable for at least 3 months. If needed, the formulation is lyophilized.

All analytical results are within the expected range:

6 mM DOTAP, 3.5 mM DOPC, 0.5 mM lipid-ICG.

### PCS: Zₐᵥₑ=170 nm, PI=0.25

### Example 3

### Preparation of EndoTAG Containing DiR

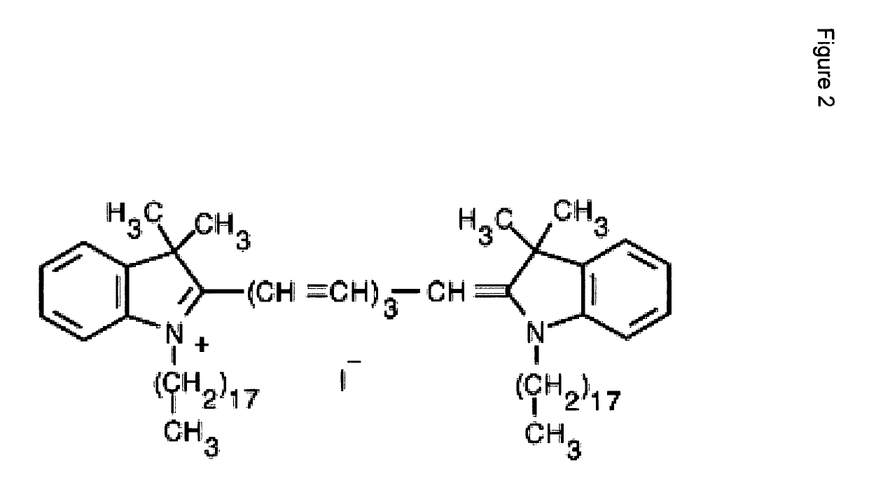

The cyanine dye DiR (excitation/emission maxima at 730/780 nm, for structure see FIG. 2) can easily be incorporated into EndoTAG since it contains two alkyl chains which associate with the lipid membrane. The free dye shows almost no fluorescence in aqueous solution but exhibits strong fluorescence in the membrane. Thus, background fluorescence due to dye lost from the liposome is negligible.

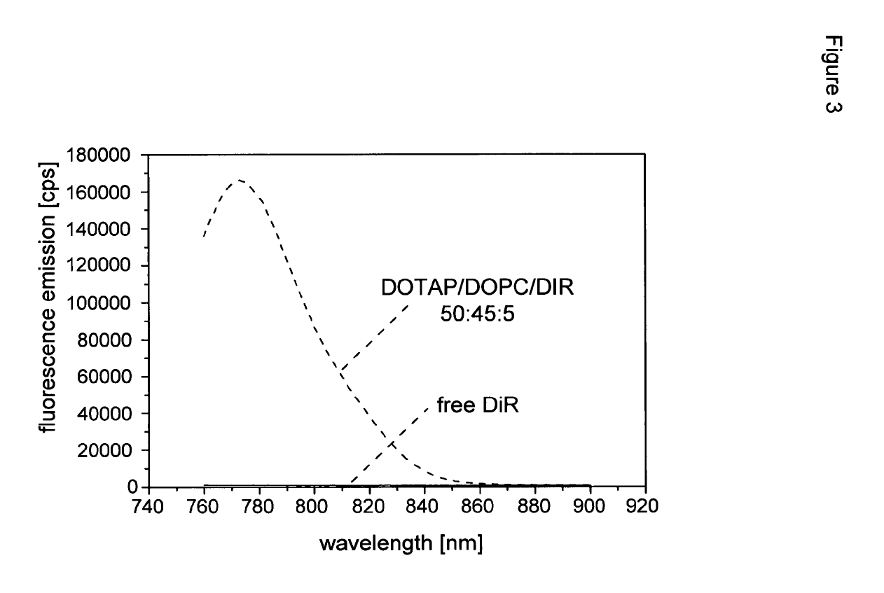

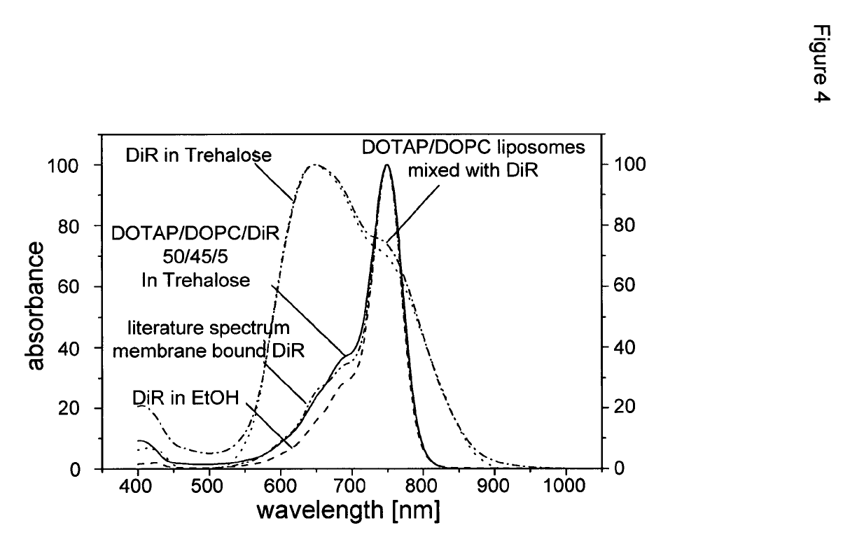

Liposomes are prepared by the lipid film method (see for example WO 2004/002468) as follows: In a round bottom flask, 0.06 mmol DOTAP, 0.035 mmol DOPC and 0.005 mmol DiR are dissolved in chloroform. Next, the solvent is evaporated under vacuum and the thin lipid film is dried for about 60 min at 100 mbar. Subsequently, 10 ml of trehalose is added to the lipid film and multilamellar vesicles (MLVs) are formed spontaneously. The total liposomal components are 10 mM (total lipid content). The MLVs are extruded five times through a polycarbonate membrane with 200 nm pore size and analysed with PCS for particle size and distribution and with HPLC for concentration of lipid as described above. Fluorescence spectroscopy is used to characterize the spectral properties of DiR in the liposome. This is illustrated in FIG. 3 and FIG. 4. FIG. 3 shows that the fluorescence intensity of the free dye in water (or trehalose as in the figure) is negligible, only upon incorporation into the lipid membrane the molecule emits fluorescence. In FIG. 4, the spectral shifts of DiR, depending on its molecular environment, are shown. The formulation is stable for at least 3 months. If needed, the formulation is lyophilized. All analytical results are within the expected range:

6 mM DOTAP, 3.5 mM DOPC, 0.5 mM DiR.

### PCS: Zₐᵥₑ=155 nm, PI=0.3

### Example 4

### Preparation of EndoTAG Labelled with Membrane Bound Fluorescent Dyes

Cationic liposomes can be labelled with other fluorescent dyes. Preferably, the dye is covalently coupled to the lipid to assure anchoring in the membrane. Suitable dyes for visualization in the VIS range can be Dansyl (336/517 nm), Marina Blue (365/460 nm), Pacific Blue (410/455 nm), NBD (463/536 nm), Fluorescein (496/519 nm), BODIPY (530/550 nm), Tetramethylrhodamine (540/566 nm), Lissamine Rhodamine (560/581 nm), BODIPY (581/591 nm), Texas Red (582/601 nm). Suitable dyes for visualization in the near-IR range are ICG or derivatives of it (790/830 nm), Alexa Fluor 790 (790/810 nm), DiR (750/800 nm).

Other dyes are possible as well. Near-IR dyes are able to visualize vessels/neovascularization beneath the RPE, i.e. in the choroid. The formulation contains a cationic lipid which amounts to 50 mol % or more in the composition. The concentration of the lipid-coupled dye is typically between 2 and 10 mol %. The remaining components of the liposome (typically 2545 mol %) can for example be DOPC, DOPE or cholesterol. Preparation and analysis of these liposomes may be performed by suspension in glucose or trehalose or another isotonic excipient which can have cryoprotecting properties in accordance to the examples described above. The liposomal preparation may be lyophilised.

### Example 5

### Optimization of Dye and Lipid Content and Zeta Potential in Cationic Liposomes

The content of cationic lipid is around 50 mol %, but the precise composition is optimized for each dye to have both optimal zeta potential (above 30 mV in 50 mM KCl solution) and optimal fluorescence properties of the dye. It has been shown that fluorescence intensity is modulated by mol % of cationic component, e.g. Table below. The data illustrates that with increasing cationic lipid, the fluorescence intensity decreases yet the zeta potential increases slightly. Based on the table below, a liposome composition of 60/35/5 (DOTAP/DOPC/Rhodamine-DOPE) was selected for in vivo work.

TABLE 1
Fluorescence intensity and charge of different formulations. The data show
that with increasing cationic lipid, fluorescence intensity decreases.

| | | | | | Fluorescence | |
|---|---|---|---|---|---|---|
| | | | mol % Liss. | Fluorescence | intensity in | |
| | mol % | mol % | Rhodamine | intensity in | methanol | zeta |
| Batch | DOTAP | DOPC | DOPE | trehalose | [cps] | potential [mV] |
| AB260 | 50 | 45 | 5 | 327820 cps, | 604864 | 48.8 |
| | | | | 100% | | |
| CF12 | 60 | 35 | 5 | 223570 cps, | 639082 | 57.3 |
| | | | | 68% | | |
| CF13 | 70 | 25 | 5 | 143026 cps, | 559682 | 66.8 |
| | | | | 44% | | |
| CF14 | 80 | 15 | 5 | 155436 cps, | 663987 | 59.3 |
| | | | | 47% | | |
| CF15 | 90 | 5 | 5 | 149195 cps, | 613899 | 62.6 |
| | | | | 46% | | |
| CF16 | 95 | 0 | 5 | 130619 cps, | 596230 | 65.8 |
| | | | | 40% | | |

### Example 6

### Preparation of EndoTAG Encapsulating a Water Soluble Fluorescent Dye or Therapeutic Agent

It is also possible to encapsulate a water soluble dye or therapeutic agent into the liposome. For encapsulation of water soluble components, DSTAP, DPTAP or DMTAP may be selected as cationic component. As neutral component, cholesterol, DSPC, DPPC, DMPC, egg PC and/or soy PC may be selected. The dye may be encapsulated in a quenching concentration. Release of the dye or the therapeutic agent can be accomplished by laser.

Specifically, liposomes are prepared by the lipid film method as follows: In a round bottom flask, 0.6 mmol DSTAP and 0.4 mmol cholesterol are dissolved in chloroform. Next, the solvent is evaporated under vacuum and the thin lipid film is dried for about 60 min at 100 mbar. Subsequently, 10 ml of an aqueous solution of water soluble dye or a therapeutic agent, e.g. 10 mM Oregon Green 488 is added to the lipid film and multilamellar vesicles are formed spontaneously. The MLVs are extruded (5×200 nm) and analysed with PCS for particle size and distribution and with HPLC for concentration of lipid and lipid-coupled dye or drug. The dye or therapeutic agent which was not encapsulated is separated from the liposomes by dialysis or cross flow. Fluorescence and UV/VIS spectroscopy are used to characterize the spectral properties of an encapsulated dye. The formulation can be lyophilized.

### Example 7

### Evaluation of EndoTAG-OG In Vivo in a Laser Induced CNV Mouse Model

For animal experiments, the laser induced CNV model in mice was used as described by Tobe et al. (1998). In brief, C57/BI6 mice (8-12 weeks old) were anesthetized, pupils were dilated and 4-6 burns of 100 μm diameter were produced with a laser (100 mW, 100 μs). In 80-90% of the laser burns, CNV develops within about 14 days.

As read-out, Scanning Laser Opthalmoscopy (SLO), flat mount and histology are performed. 100 μl of the respective formulation was applied intravenously, immediately followed by SLO over a time course of about 120 min. Three different EndoTAG formulations are selected:

EndoTAG-A: DOTAP/DOPC/OG-DHPE=60/35/5 mol %, 10 mM total liposomal components in 10% trehalose (produced as described in Example 1).
EndoTAG-B: DOTAP/DOPC/OG-DOPE=9/5/5 mol %, 10 mM total liposomal components in 10% trehalose
EndoTAG-C: DOTAP/DOPC/OG-DOPE/PEG-DOPE=60/30/5/5 mol %, 10 mM total liposomal components in 10% trehalose

As control, neutral liposomes (DOPC/OG-DOPE=95/5 mol %, 10 mM total lipid, in trehalose) is applied as well as an aqueous solution of Oregon Green (0.5 mM). Additional animals are used to carry out flat mounts and histology by sacrificing animals at the respective peak intensity as observed in SLO.

### SLO Results

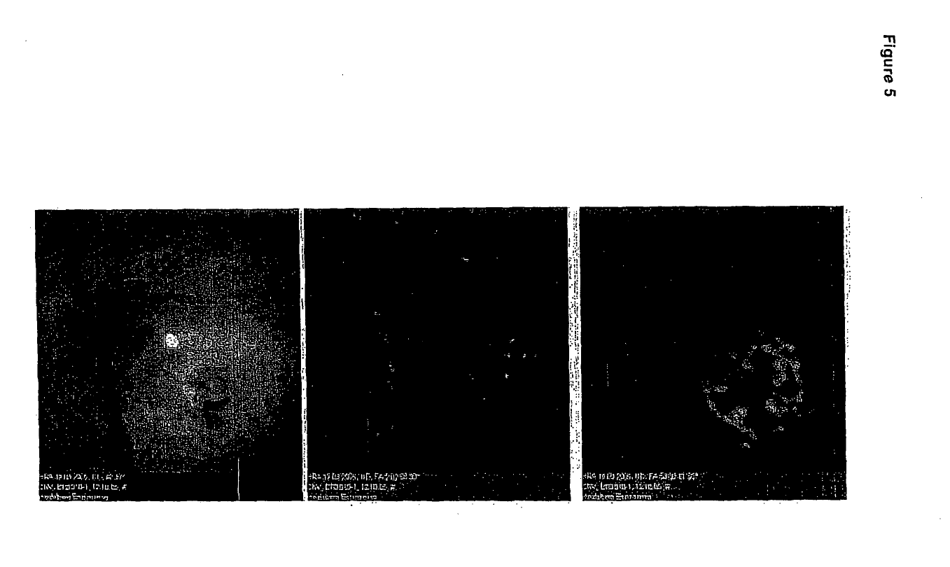

With EndoTAG-A and B, after about 30 min, specific enrichment at the lesion site is visualized (see FIG. 5). This signal persists until about 80 min after application, having a maximum at about 50-70 minutes.

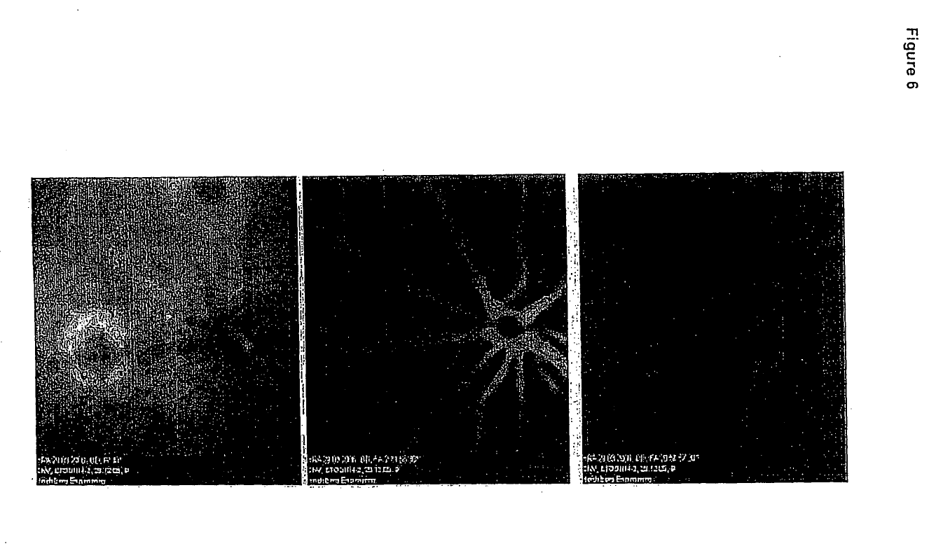

The neutral liposomes showed enhancement of the vasculature for about 30-50 min. However, no accumulation in lesion site was seen (see FIG. 6). At the equivalent dye concentration, the free dye did not show an accumulation.

### Example 8

### Preparation of Cationic Liposomes Comprising Paclitaxel (EndoTAG™-1)

A cationic liposomal preparation comprising DOTAP, DOPC and paclitaxel in a ratio of about 50:47:3 and a lipid content of 10 mM in a 10% m/m trehalose dihydrate solution is prepared according to the method disclosed in WO 2004/002468. The respective preparation is designated as EndoTAG™-1.

Briefly, DOTAP-chloride, DOPC and paclitaxel are dissolved in ethanol to a concentration of 400 mM of total lipophilic compounds. Liposomes are subsequently generated by the ethanol injection method by injection into a trehalose solution. The liposomal dispersion is extruded five times through a 200 nm polycarbonate membrane.

The final liposomal preparation is sterile filtered through a 0.22 μm membrane and lyophilized for storage.

Prior to use in animal studies, the lyophilized powder is reconstituted with water for injection.

### Example 9

### Preparation of Cationic Liposomal Preparation Comprising Methotrexate (MTX)

20 mM DOTAP liposomes (20 ml) are prepared by the lipid film method as described in WO 2004/002468, rehydration is performed with 10% trehalose. Next, the liposomes are mixed with 20 ml of a sodium MTX solution (2.2 mM, prepared from diluting a 220 mM sodium MTX solution with 10% trehalose). The resulting solution (theoretical concentration now 10 mM DOTAP and 1.1 mM MTX) is extruded 5 times through a polycarbonate membrane with 200 nm pore size.

Subsequently, HPLC and PCS analytics are performed. DOTAP: 8.4 mM, MTX 1.14 mM (for HPLC methods, see below). Zₐᵥₑ=156 nm, PI 0.29

Other analytics: zeta potential: 59.3 mV±1.4 mV (after 1:10 dilution in a solution containing 50 mM KCl and 10% trehalose)
MTX release from liposome is determined by centrifugation through Centricon tube, (MWCO=30,000, 4500 rcf, 180 min) and is found to be 1.4% of the MTX concentration.

The formulation is stable at 4° C. for at least 16 weeks.

### Example 10a

### Evaluation of Cationic Liposomal Preparations Comprising a Therapeutically Active Agent in an In Vivo Laser Induced CNV Mouse Model

The therapeutic potential of EndoTAG™-1 and other cationic liposomal preparations comprising an therapeutically active agent can be evaluated in a laser induced CNV model in mice according to Tobe et al. (Tobe et al., 1998) as described above.

Animals are assigned to different treatment groups and given i.v. doses of EndoTAG™-1 covering a dose range from 1.28 mg paclitaxel/kg body weight up to 10 mg/kg per application and covering a cumulative dose range from 12 mg/kg to 50 mg/kg.

Application of drug is started on day 1 after laser wounding. The drug is applied over 1-2 weeks, 2-4 times weekly.

The effect of the treatment can be assessed by the analytical methods described in Example 8.

### Example 10b

### Evaluation of EndoTAG™-1 Vs. Taxol® in the Laser Induced CNV Mouse Model

For animal experiments, the laser induced CNV model in mice was used as described by Tobe et al. (1998). In brief, C57/BI6 mice (8-12 weeks old) were anesthetized, pupils were dilated and 4 burns of 75 μm diameter were produced with a laser (150 mW, 100 μs).

Animals were assigned to different treatment groups (n=8) which received either EndoTAG™-1, Taxol® or no treatment. Treatment started on day 0 and was repeated on day 2, 4, 7, 9, 11, 14, 16 for a total of 8 treatments. EndoTAG™-1 and Taxol® were administered at a dose of 2.56 mg/kg paclitaxel. SLO using fluorescein (2.5 μl/g body weight of a 10% sodium fluorescein solution) was performed on day 10, 15 and 17. All SLO images were evaluated 5 min after fluorescein application according to the grading system of Takehana et al. (Takehana et al., 1999). In brief, the following scores will be given in a masked fashion by two different examiners:
score 0: no leakage
score 1: slightly stained
score 2: moderately stained
score 3: strongly stained

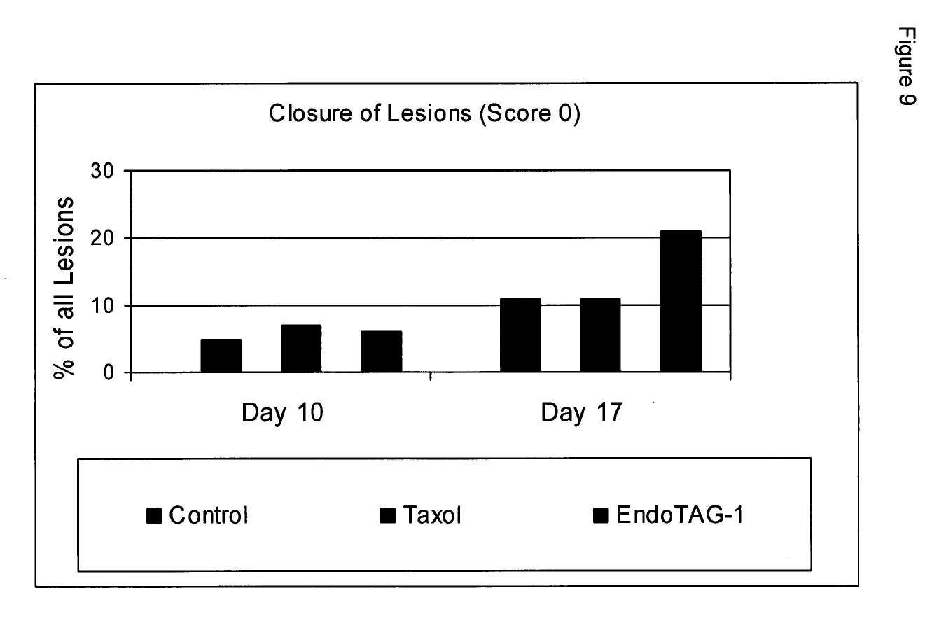

The results of the experiment are shown in FIG. 9. The figure displays the closure of lesions expressed as percentage of nonleaky lesions (score 0) of all lesions in the three groups. Whereas on day 10 this percentage was still comparable in all three groups, it increased dramatically only in the EndoTAG-1 group on day 17. Thus, a therapeutic effect of EndoTAG™-1 in an in vivo CNV animal model is demonstrated.

### Example 10c

### EndoTAG™-Spa and EndoTAG™-1 Vs. Taxol® in the Laser Induced CNV Mouse Model

For animal experiments, the laser induced CNV model in mice was used as described by Tobe et al. (1998). In brief, C57/BI6 mice (8-12 weeks old) were anesthetized, pupils were dilated and 4 burns of 75 μm diameter were produced with a laser (150 mW, 100 μs).

EndoTAG™-1 was prepared as described in Example 8, cationic liposomes comprising succinyl paclitaxel comprising DOTAP/DOPC/succinyl paclitaxel in 50/39/11 mol % (EndoTAG™-SPA) were prepared according to Haas et al., WO 2004/002455.

Animals were assigned to different treatment groups (n=4) as follows:
EndoTAG-1, 0.5 mg/kg paclitaxel dose
EndoTAG-1, 2.5 mg/kg paclitaxel dose
EndoTAG-SPA, 0.5 mg/kg paclitaxel dose
EndoTAG-SPA, 2.5 mg/kg paclitaxel dose
Taxol, 0.5 mg/kg paclitaxel dose
Taxol, 2.5 mg/kg paclitaxel dose
trehalose (control)

After laser wounding (day 0), animals received the respective intravenous treatment on day 1, 3, 5, 7 and 9. On day 10, animals were perfused with FITC dextran, eyes were enucleated and flat mounts of sclera, choroid and RPE were prepared. The flat mounts were analysed with fluorescence microscopy and the area of FITC-dextran perfused blood vessels in each individual lesion was quantified by two independent and blinded evaluators. Quantification was performed with Image J software.

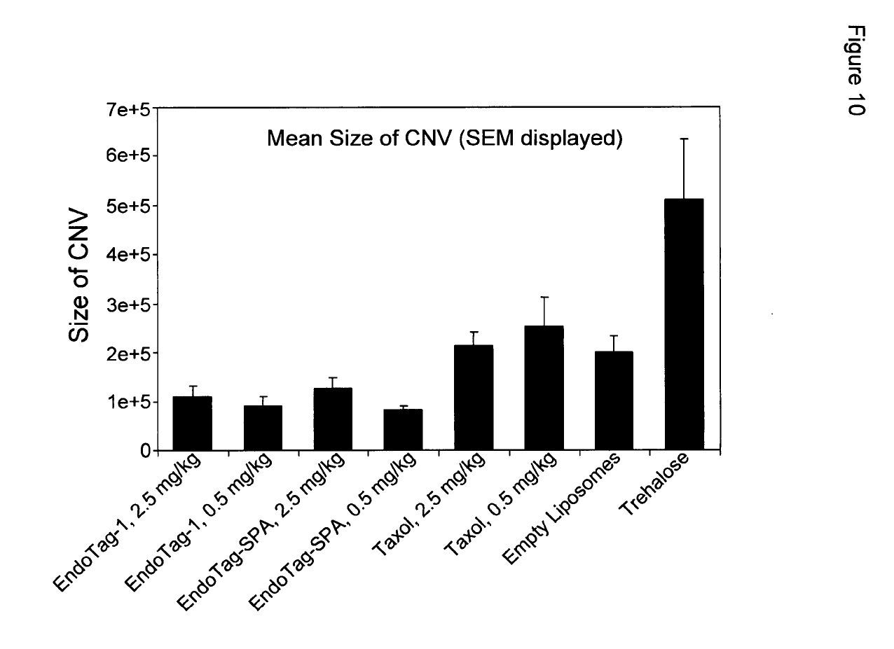

The results of the study are depicted in FIG. 10. The data show that all EndoTAG™ based therapeutics inhibited development of CNV to a degree which was statistically significant vs. trehalose group Remarkably, in the EndoTAG™-1 and EndoTAG™-SPA groups, the lower dosing schedule of 0,5 mg/kg showed a therapeutic effect that was slightly greater than the effect of the 2,5 mg/kg dosing schedule.

### Example 11

### Preparation of Cationic Liposomes Comprising a Photosensitizer

Photosensitizers can be encapsulated in cationic liposomes. The photosensitizer can be embedded in the membrane, encapsulated in the aqueous interior or covalently coupled to the liposome membrane.

Suitable molecules for membrane embedding are haematoporphyrin, protoporphyrin IX, Photofrin or other porphyrin or benzoporphyrin derivatives, phthalocyanine derivatives, chlorin, purpurin, texaphyrin, indocyanine (ICG). A suitable molecule for encapsulation into the aqueous interior of the liposome is ALA (5-aminolevulinic acid). The ALA hexyl ester or another lipid-coupled form of ALA can be attached to the liposome membrane. For the preparation of liposomes for the treatment of occult CNV, a photosensitizer with an absorbance in a long wavelength, such as ICG, is preferably selected.

The formulation contains a cationic lipid which amounts to 50 mol % or more in the composition. The concentration of the photosensitizer is typically between 2 and 20 mol %. The remaining components of the liposome (typically 25-45 mol %) can for example be DOPC, DOPE or cholesterol. Preparation and analysis of these liposomes may be performed by suspension in glucose or trehalose or another isotonic excipient which can have cryoprotecting properties in accordance to the examples described above. The preparation of liposomes according to the “lipid film method” or “ethanol injection method” is also described in WO 2004/002468.

### Example 12

### Preparation of Cationic Liposomes Comprising Verteporfin

The benzoporphyrin derivative verteporfin (USP Material, Catalog number 1711461) is encapsulated in a liposomal preparation composed of DOTAP and DOPC. The molar composition DOTAP/DOPC/verteporfin is x/95-x/5 or y/90-y/10 with x varying between 50 and 95 and y varying between 50 and 90.

The components are dissolved in chloroform in a round bottom flask, then the chloroform is evaporated under vacuum (about 60 min, 100 mbar) and the resulting lipid film is rehydrated in trehalose (10%). Multilamellar vesicles (MLVs) are formed spontaneously and the resulting overall concentration of lipids and verteporfin is 10 mM. The MLVs are extruded five times through a polycarbonate membrane with 200 nm pore size. The resulting SUVs (small unilamellar vesicle) are analysed with PCS for particle size and size distribution and with HPLC for concentration of lipid (as described above) and photosensitizer. Fluorescence and UV/VIS spectroscopy are used to characterize the spectral properties of verteporfin.

### Example 13

### Evaluation of Cationic Liposomes Comprising a Photosensitizer In Vitro

Human macrovascular umbilical vein endothelial cells (HUVEC) with no more than 4 passages are grown in vitro in complete endothelial cell basal medium supplemented with 5% fetal bovine serum. HUVEC are propagated in Roux flasks coated with 1.5% bovine skin gelatin type B.

Incubation of HUVEC with a cationic liposomal preparation comprising the photosensitizer verteporfin (5 mol %) as described in Example 12.1 is carried out into 96-well plates coated with gelatin. As control, the photosensitizer is formulated in neutral (100% DOPC, 100% DMPC) or negatively charged liposomes (DMPC/EPG=65/35 mol %), respectively. These control formulations are also added to the cells.

After incubation with photosensitizer for 5 minutes, the cells are washed, medium is replaced and the cells are irradiated with doses between 10 and 150 J/cm² using a diode laser system with a wavelength of 690 nm. 24 hours after irradiation, cells are assayed for viability.

### Example 14

### Evaluation of a Cationic Liposomal Preparation Comprising Verteporfin in in an In Vivo Laser Induced CNV Mouse Model

The therapeutical effect of photosensitizers encapsulated in liposomes is assessed in a laser induced CNV mouse model (as above). After the intravenously application of the liposomal preparation, the photosensitizer is activated by a laser treatment of the eye.

### Example 15

### In Vitro Determination of the Inhibitory Activity of EndoTAG™-1 and EndoTAG™-Placebo on IL-6- and IL-8-Release from HUVEC Stimulated with TNFalpha

The pro- and/or anti-inflammatory activity of liposomes formulations such as EndoTag™-1 and EndoTag-placebo on endothelial cells can be assessed by analysing inflammatory cytokines that are released by HUVEC in the growth culture medium after treatment with these drugs. The higher the anti-inflammatory activity of these liposomes, the lower is the amount of IL-6 and IL-8 release from stimulated cells.

### Experiment:

Primary HUVEC (passages 2 to 4; 1×10⁴ cells/well) were grown overnight into 500 μl EGM2 full medium (Endothelial growth cell medium containing 5% FBS) in 24 well plates. Culture medium was removed and 500 μl of fresh cultured medium containing TNFα (30 U/ml) and 1, 50, 100 or 500 nM EndoTAG™-1 (DOTAP 50%/DOPC 47%/paclitaxel 3%) or EndoTAG™-placebo (DOTAP 50%/DOPC 50%) either in EGM2 full medium or in EGM2 low medium (Endothelial growth cell medium containing 0.5% FBS) was added. A control sample was treated with medium which did not comprise TNFα. EndoTAG™-1 was prepared by the injection method as described in WO 2004002468 by Mundus et al. EndoTAG™-placebo was prepared accordingly. The supernatant was harvested after 48 hrs and the amounts of IL-8 and IL-6 cytokines were measured using the “BD Cytometric Bead Array”. Measurement was done in triplicates.

### Result:

Stimulation of HUVEC with physiologically relevant concentration of inflammatory cytokines such as TNFα (30 U/ml) in presence of increasing concentration of EndoTAG™-1 or EndoTAG™-placebo clearly show an inhibition of IL-6 and IL-8 released by HUVEC. Notably, at the lowest concentration of EndoTag-1 used in this assay (1 nM EndoTAG™-1) 37% inhibition of IL-8 (FIG. 4, right part) and 35% II-6 (FIG. 4, left part) release can be observed. A concentration of 50 nM of EndoTAG™-1 was sufficient to get up to 71% inhibitory activity whereas a ten fold higher concentration of EndoTAG™-1 (up to 500 nM) does not show a significant increase of the inhibitory activity as compare to 50 nM concentration (FIG. 4). Under the same experimental conditions EndoTAG™-placebo shows up to 34% inhibition of IL-8 (FIG. 4, right part) or 21% II-6 (FIG. 4, left part) release by HUVEC

### Example 16

### Therapeutic Effect of EndoTAG-1 on Rat Carrageenan-Induced Paw Inflammation

Male Sprague Dawley rats with an average weight of 248 grams at arrival were purchased from Harlan Inc. and housed in isolated cages under save environmental conditions (3-4 rats per cage, 22° C., 30-70% humidity and 12 h light/dark cycle) with food and water ad libitum. Animals were acclimated for 3 days prior to being placed on study. Experimental design was reviewed and approved by local government.

Animals (6/group), were injected with 100 μl of 1.2% Carrageenan into the right hind footpad and were then euthanized at four hours post injection for evaluation of paw swelling, based on paw weight determination. EndoTag-1 (DOTAP 50%/DOPC 47%/paclitaxel 3%) or EndoTag-placebo (DOTAP 50%/DOPC 50%) with a lipid content of 10 mM in a 10% m/m trehalose dihydrate solution were prepared as described in WO 2004/002468 by Mundus et al. Taxol® was a CremophorEL formulated Paclitaxel purchased from Bristol-Myers Squibb. Drug solutions were administered iv with slow bolus in a volume of 10 μl/g into the tail vein. Animals were dosed 30 minutes post Carrageenan injection as indicated below:

| | Animal | | Appl. vol. | |
|---|---|---|---|---|
| Group name | no. | Treatment | [ml/kg] | Dose mg/kg |
| Disease control | 6 | 9.8% | 5 | — |
| | | Trehalose | | |
| EndoTAG placebo | 6 | EndoTAG-1 | 5 | — |
| | | placebo | | |
| Taxol ® | 6 | Taxol | 5 | 1.28* |
| EndoTAG-1 | 6 | EndoTAG-1 | 5 | 1.28* |

*given as paclitaxel concentration

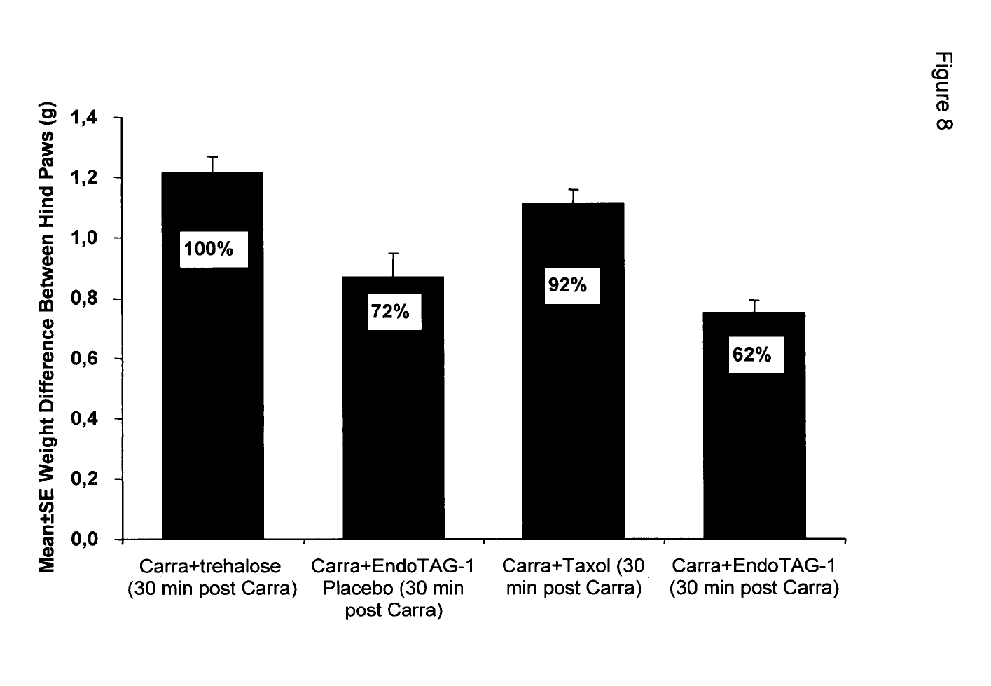

FIG. 8 shows that treatment with EndoTAG-1 or EndoTAG placebo had a significant therapeutic effect on rat Carrageenan-induced paw inflammation, measured as decrease in paw weight. Treatment with EndoTAG-1 30 minutes post Carrageenan injection significantly reduced left/right (untreated/inflamed) paw weight differences compared to the post-injection trehalose group by 38%, as well as reducing the weight difference compared to the Taxol® group by 33%. EndoTAG placebo treatment significantly reduced weight difference by 28%, while Taxol® did not show any effect. The effects of Taxol® given as 1.28 mg/kg/day paclitaxel alone and EndoTAG-1 placebo alone were generally additive for the EndoTAG-1 given as 1.28 mg/kg/day paclitaxel.

### REFERENCES

Adamis, A. P., Aiello, L. P., and D'Amato, R. A. (1999). Angiogenesis and ophthalmic disease. Angiogenesis 3, 9-14.
Bressler, N. M., and Bressler, S. B. (2000). Photodynamic therapy with verteporfin (Visudyne): impact on opthalmology and visual sciences. Invest Opthalmol Vis Sci 41, 624-628.
Campochiaro, P. A. (2000). Retinal and choroidal neovascularization. J Cell Physiol 184, 301-310.
Chen, K. H., Wu, C. C., Roy, S., Lee, S. M., and Liu, J. H. (1999). Increased interleukin-6 in aqueous humor of neovascular glaucoma. Invest Opthalmol Vis Sci 40,:2627-2632.
Ebrahim, S., Peyman, G. A., and Lee, P. J. (2005). Applications of liposomes in opthalmology. Surv Opthalmol 50, 167-182.
Higgins, G. T., Wang, J. H., Dockery, P., Cleary, P. E., and Redmond, H. P. (2003) Induction of angiogenic cytokine expression in cultured RPE by ingestion of oxidized photoreceptor outer segments. Invest Opthalmol Vis Sci. 44, 1775-1782.
Holz, F. G., Pauleikhoff, D., Spaide, R. F., Bird, A. C., (2004) Age-related macular degeneration. Berlin Heidelberg, Springer-Verlag, 93-94.
Hosokawa, T., Sami, M., Kato, Y., and Hayakawa, E. (2003). Alteration in the temperature-dependent content release property of thermosensitive liposomes in plasma. Chem Pharm Bull (Tokyo) 51, 1227-1232.
Kimura, H., Yasukawa, T., Tabata, Y., and Ogura, Y. (2001). Drug targeting to choroidal neovascularization. Adv Drug Deliv Rev 52, 79-91.
Moshfeghi, A. A., and Peyman, G. A. (2005). Micro- and nanoparticulates. Adv Drug Deliv Rev 57, 2047-2052.
Needham, D., Anyarambhatla, G., Kong, G., and Dewhirst, M. W. (2000). A new temperature-sensitive liposome for use with mild hyperthermia: characterization and testing in a human tumor xenograft model. Cancer Res 60, 1197-1201.
Olejnik, O., and P., H. (2005). Drug delivery strategies to treat age-related macular degeneration. Adv Drug Deliv Rev 57, 1991-1993.
Peyman, G. A., Moshfeghi, D. M., Moshfeghi, A. A., and Khoobehi, B. (1996). Fluorescent vesicle angiography with sodium fluorescein and indocyanine green. Ophthalmic Surg Lasers 27, 279-284.
Rabinovich-Guilatt, L., Couvreur, P., Lambert, G., and Dubernet, C. (2004). Cationic vectors in ocular drug delivery. J Drug Target 12, 623-633.
Ratner, M. (2004). Genentech discloses safety concerns over Avastin. Nat. Biotechnol 22, 1198.
Takehana, Y., Kurokawa, T., Kitamura, T., Tsukahara, Y., Akahane, S., Kitazawa, M., Yoshimura, N. 1999. Suppression of laser-induced choroidal neovascularization by oral tranilast in the rat. Invest Opthalmol Vis Sci 40, 459-466.
Tobe, T., Ortega, S., Luna, J. D., Ozaki, H., Okamoto, N., Derevjanik, N. L., Vinores, S. A., Basilico, C., and Campochiaro, P. A. (1998). Targeted disruption of the FGF2 gene does not prevent choroidal neovascularization in a murine model. Am J Pathol 153, 1641-1646.
van Wijngaarden, P., Coster, D. J., and Williams, K. A. (2005). Inhibitors of ocular neovascularization: promises and potential problems. Jama 293, 1509-1513.
Yasukawa, T., Ogura, Y., Sakurai, E., Tabata, Y., and Kimura, H. (2005). Intraocular sustained drug delivery using implantable polymeric devices. Adv Drug Deliv Rev 57, 2033-2046.
Yoshida, A., Yoshida, S., Khalil, A. K., Ishibashi, T., and Inomata H. (1998) Role of NF-kappaB-mediated interleukin-8 expression in intraocular neovascularization. Invest Opthalmol V is Sci. 39, 1097-106.
Zhou, J., Pham, L., Zhang, N., He, S., Gamulescu, M. A., Spee, C., Ryan, S. J., and Hinton, D. R. (2005). Neutrophils promote experimental choroidal neovascularization. Mol. Vis. 16, 414-424.

1. A method of selectively delivering at least one active agent to the angiogenic sites of neovascular ocular endothelium comprising the systemic administration of a cationic colloidal preparation comprising at least one active agent.

2. A method of treating, preventing or diagnosing an ocular neovascularization disease comprising the systemic administration of a cationic colloidal preparation comprising at least one active agent.

3. The method of claim 1, wherein said cationic colloidal preparation comprises a positive zeta potential.

4. The method of claim 1, wherein said cationic colloidal preparation comprises a cationic liposome.

5. The method of claim 1, wherein said active agent is a therapeutic agent, a diagnostic agent or a combination comprising a therapeutic and a diagnostic agent.

6. A method for preventing, treating and/or diagnosing an ocular neovascularization comprising systemical administration of a cationic colloidal preparation comprising at least one active agent.

7. The method of claim 6, wherein said active agent is a therapeutic agent, a diagnostic agent or a combination comprising a therapeutic and a diagnostic agent.

8. The method of claim 5, wherein said therapeutic agent is an antiangiogenic agent.

9. The method of claim 5, wherein said therapeutic agent is a cytotoxic or cytostatic agent, preferably a antineoplastic agent especially antimitotic agent like a taxane, an anthracyclin preferably doxorubicin or epirubicin, a statin, a depsipeptide, thalidomide, another agent interacting with microtubuli such as discodermolide, laulimalide, isolaulimalide, eleutherobin, epothilone, Sarcodictyin A and B, an antimetabolite preferably an antifolate, an alkylating agent especially a platinum containing compound like cisplatin or carboplatin, a DNA topoisomerase inhibiting agent like camptothecin, an RNA/DNA antimetabolite, especially 5-fluorouracil, gemcitabine or capecitabine.

10. The method of claim 9, wherein said taxane is paclitaxel, docetaxel, on any derivative thereof.

11. The method of claim 5, wherein said therapeutic agent is an antagonist of a growth factor like VEGF, PDGF, EGF, FGF, preferably an antagonist of VEGF.

12. The method of claim 11, wherein said antagonist of VEGF is an antibody or antibody fragment like bevacizumab or rhufab V2, a soluble receptor or a fusion protein with receptor fragments like VEGF-TRAP_{R1R2}, a growth factor receptor kinase inhibitor, a protein kinase C inhibitor, a nucleic acid based antagonist like an siRNA against VEGF or VEGFR-1 or 2, or an aptamer like pegaptanib sodium.

13. The method of claim 5, wherein said therapeutic agent is an anti-inflammatory agent such as a synthetic glucocorticoid, mineralocorticoid, hydrocortisone, dexamethasone, fluocinolone, prednisone, prednisolone, methylprednisolone, fluorometholone, betamethasone and triamcinolone, a non-steroidal anti-inflammatory agent such as salicylate, indomethacin, ibuprofen, diclofenac, flurbiprofen, piroxicam or a COX2 inhibitor.

14. The method of claim 5, wherein said therapeutic agent is an antagonist against cellular adhesion molecules, preferably an antibody directed against alpha5 beta1 integrin, alpha5 beta3 integrin or alpha5 beta5 integrin or a RGD peptide.

15. The method of claim 5, wherein said therapeutic agent is a cytokine like interferon or an interleukin or a chemokine.

16. The method of claim 5, wherein said therapeutic agent is a photosensitizer, preferably a porphyrin or a precursor or derivative of a porphyrin.

17. The method of claim 16, wherein said porphyrin is a green porphyrin, preferably a derivative of hydro-mono benzoporphryins.

18. The method of claim 5, wherein said diagnostic agent is a diagnostically detectable label, preferably a fluorescent label, a histochemical label, an immunohistochemical label, a radioactive label, or a contrast agent for MRI, CT and/or X-ray.

19. The method of claim 18, wherein said fluorescent label is a fluorescence dye in the visual and near-infrared wavelength range, preferably fluorescein or a derivative like 6-carboxy-fluorescein, Oregon Green or a derivative, Pacific Blue, a rhodamine dye, especially Lissamine Rhodamine, Alexa Fluor 790, or a cyano dye like indocyanine green (ICG) or, DiR or a derivative.

20. The method of claim 18, wherein said fluorescence dye is detected by scanning laser opthalmoscopy.

21. A method of reducing the release of pro-inflammatory cytokines in the course of an ocular neovascularization disease, comprising the administration of a cationic colloidal carrier preparation, which preferably comprises a therapeutic agent.

22. The method of claim 21 wherein the pro-inflammatory cytokine is selected from IL-6 and/or IL-8.

23. A method of reducing inflammation in the course of an ocular neovascularization disease, comprising the administration of a cationic colloidal carrier preparation.

24. A method for the treatment of inflammation in the course of an ocular neovascularization disease comprising preparing a cationic colloidal carrier preparation.

25. The method of claim 1, wherein said ocular neovascularization disease is macular degeneration, preferably age related macular degeneration or retinopathy, preferably proliferative diabetic retinopathy.

26. The method of claim 1, wherein said systemic administration is intravenous administration.

27. The method of claim 1, wherein said preparation is administered to a human patient.

28. The method of claim 1, wherein said colloidal carrier preparation is a liposomal preparation.

29. The method of claim 1, wherein said cationic colloidal carrier preparation comprises a cationic lipid, optionally at least one further amphiphile and optionally at least one stabilizing agent.

30. The method of claim 1, wherein said cationic colloidal carrier preparation comprises a cationic lipid in an amount of at least about 30 mol % of total lipid, and optionally at least one further amphiphile in an amount of up to about 70 mol % of total lipid.

31. The method of claim 29, wherein said further amphiphile is a neutral and/or anionic lipid.

32. The method of claim 29, wherein said cationic lipid is DOTAP, DSTAP₁ DMTAP and/or DPTAP, preferably DOTAP.

33. The method of claim 31, wherein said neutral lipid is a phosphatidylcholine (PC), preferably DOPC, DSPC, DPPC, DMPC, egg PC and/or soybean PC.

34. The method of claim 1, wherein said colloidal carrier preparation comprises unilamellar liposomes.

35. The method of claim 1, wherein said colloidal carrier preparation comprises liposomes with an average particle size of about 50 nm to about 400 nm, preferably about 100 nm to about 300 nm.

36. The method of claim 1, wherein said cationic colloidal carrier preparation comprises a positive zeta potential when measured in about 0.05 mM KCl solution at about pH 7.5.

37. The method of claim 36, wherein said positive zeta potential is greater than about 20 mV, preferably greater than about 40 mV.

38. The method claim 1, wherein the preparation further comprises a pharmaceutically acceptable carrier, diluent or adjuvant.

39. A composition comprising a cationic colloidal carrier preparation comprising a near-infrared fluorescent dye, or fluorescein, or derivative as an active agent.

40. The composition of claim 39, wherein said near-infrared dye is selected from indocyanine green (ICG) and derivatives, Alexa Fluor 790, dioctadecyltetramethyl indotricarbocyanine Iodide (DIR) and derivatives.

41. The composition of claim 39, wherein said cationic carrier preparation is a liposomal preparation comprising a cationic lipid in an amount of at least about 50 mol %, optionally at least one further amphiphile in an amount of up to about 50 mol % and a near-infrared fluorescent dye in an amount of up to about 20 mol %.

42. The composition of claim 41, wherein said further amphiphile is a neutral and/or anionic lipid.

43. The composition of claim 41, wherein said cationic lipid is DOTAP, DSTAP, DMTAP and/or DPTAP, preferably DOTAP.

44. The composition of claim 41, wherein said neutral lipid is a phosphatidylcholine (PC), preferably DOPC, DSPC₁ DPPC, DMPC, egg PC and/or soybean PC.

45. The composition of claim 39, comprising PEG or a derivative thereof, particularly a pegylated neutral and/or anionic lipid.

46. The composition of claim 45, wherein said lipid is a pegylated phosphoethanolamine (PE) such as DOPE and/or DSPE.

47. The composition of claim 39, wherein said colloidal carrier preparation comprises liposomes with an average particle size of about 50 nm to about 400 nm, preferably about 100 nm to about 200 nm.

48. The composition of claim 39, wherein said cationic colloidal carrier preparation comprises a positive zeta potential when measured in about 0.05 mM KCl solution at about pH 7.5.

49. The composition of claim 48, wherein said positive zeta potential is greater than about 20 mV, preferably greater than about 40 mV.

50. The composition of claim 39 for diagnostic use.

51. The composition of claim 39 which additionally comprises a therapeutic agent.

52. A composition comprising a cationic colloidal carrier preparation comprising a VEGF antagonist as an active agent.

53. A composition comprising a cationic colloidal carrier preparation comprising an antagonist against cellular adhesion molecules as an active agent.

54. A composition comprising a cationic colloidal carrier preparation comprising a photosensitizer as an active agent.

55. A composition comprising a cationic colloidal carrier preparation comprising a siRNA molecule as an active agent.

56. A composition comprising a cationic colloidal carrier preparation comprising an aptamer as an active agent.

57. The composition of claim 51 for therapeutic use.
